(19) United States
(12) Patent Application Publication
(10) Pub. No.: US 2021/0183467 A1
(43) Pub. Date: Jun. 17, 2021

(54) SYSTEM AND METHOD FOR USING MICROBIOME TO DE-RISK DRUG DEVELOPMENT

(71) Applicant: YALE UNIVERSITY, New Haven, CT (US)

(72) Inventors: Andrew GOODMAN, Guilford, CT (US); Michael ZIMMERMANN, New Haven, CT (US); Maria ZIMMERMANN, New Haven, CT (US)

(73) Assignee: YALE UNIVERSITY, New Haven, CT (US)

(21) Appl. No.: 17/257,394

(22) PCT Filed: Jun. 28, 2019
(86) PCT No.: PCT/US19/39717

§ 371 Date: Dec. 31, 2020

Related U.S. Application Data
(60) Provisional application No. 62/693,741, filed on Jul. 3, 2018.

(51) Int. Cl. G16B 20/50; G16B 40/20; G16B 40/30; G16C 20/70; G16B 20/10

(52) U.S. Cl. CPC G16B 20/50; G16B 40/20; G16B 20/10; G16C 20/70; G16B 40/30

(57) ABSTRACT

The invention provides a system and method to quantitatively disentangle host and microbiome contributions to drug metabolism. The system includes a non-transitory storage medium storing information of levels of parent drug and drug metabolites for a plurality of oral drugs, by each of a plurality of microbiotas and by each of a plurality of genome-sequenced microbes in pure culture. A processor executes a predictor module which implements a computational model to quantitatively disentangle host and microbiota contributions to drug metabolism, predict how a person's microbiome will metabolize a drug candidate, predict how the metabolization impacts the drug candidate and metabolite exposure in circulation, and predict whether the drug candidate will be metabolized by a microbiota.

### CROSS-REFERENCE TO RELATED APPLICATION

This application claims priority from U.S. Provisional Application No. 62/693,741, filed Jul. 3, 2018, which is hereby incorporated by reference in its entirety.

### STATEMENT AS TO FEDERALLY SPONSORED RESEARCH

This invention was made with government support under grants GM118159, GM105456 and AI124275 awarded by the National Institutes of Health. The government has certain rights in the invention.

### FIELD OF THE INVENTION

This invention is directed to high-throughput methods and databases for testing and predicting whether and how a drug candidate will be metabolized by microbiota and identifying a subpopulation of patients for the drug administration as well as predicting drug-drug interactions.

### BACKGROUND

Drug development is an enormously expensive and time-consuming process, exceeding $1.4 billion and 10 years per successful drug. A major contributor to this expense is that promising drug candidates fail in large-scale clinical trials. These costs slow drug development and directly impact patients.

Individuals can vary widely in drug response. Most drugs are delivered orally (many in delayed release formulations), and over 70% exhibit low solubility, low permeability, or both as described in A. Dahan, J. M. Miller, G. L. Amidon, Prediction of solubility and permeability class membership: provisional BCS classification of the world's top oral drugs, AAPS J. 11, 740-746 (2009).

These drugs transit the gastrointestinal tract prior to absorption, where they encounter commensal microbes at densities exceeding 10⁸ cells/mL in the small intestine and 10¹¹ cells/mL in the colon, according to R. Sender, S. Fuchs, R. Milo, Revised Estimates for the Number of Human and Bacteria Cells in the Body, Plos Biol. 14, e1002533 (2016). These microbes collectively encode 150-fold more genes than the human genome; between individuals, microbiome variation far exceeds genome variation.

Anecdotal examples of drug-microbiome interaction have been reported in humans and mice, including drugs for inflammatory disease (e.g., Sulfasalazine; azoreduction by the microbiota), gastrointestinal disorders (Bisacodyl; ester hydrolysis), osteoporosis (Calcitonin; amide hydrolysis), and thrombosis (Sulfinpyrazone; sulfoxide reduction). In the case of the cardiac drug Digoxin and several others, microbial metabolism is highly species-specific, which has important implications in light of the enormous interpersonal variability that exists in microbiome composition. Interpersonal variation in drug metabolism has important consequences, including lack of clinical improvement, dangerous adverse reactions, and delayed drug development. As a result, extensive efforts have been made to predict the activities of drug metabolizing enzymes (DMEs) in the liver.

However, our understanding of microbiome-mediated drug metabolism is limited to a few anecdotal examples. We are currently not able to predict whether and how a drug will be metabolized by the gut microbiota, how interpersonal microbiome variation will impact this activity, or how microbiome-mediated drug metabolism impacts serum drug and metabolite exposure over time. There are no methods available to identify drug-targeting microbes or microbe-targeted drugs. Most microbiome genes have no known function.

Existing approaches for testing and predicting drug metabolism (computational or experimental) address host activities but do not provide any information about the microbiota. Prior art makes these predictions based solely on host activities and do not provide any information about the microbiota. Prior art identifies drug-metabolizing host enzymes but does not provide information about drug-metabolizing microbiota taxa. Prior art identifies only host genes that metabolize drugs. Prior art focuses on human genome polymorphisms (e.g. point mutations in CYP450-family proteins) but does not provide any information on microbiota contributions. Prior art only focuses on host-produced drug metabolites. The existing technology currently cannot predict whether an individual's microbiota predisposes them to efficient or inefficient metabolism of almost any drug. Dissecting host and microbial contributions to drug metabolism is challenging, particularly in cases where host and microbiome carry out the same metabolic transformation. Cryptic microbial contributions to drug metabolism, in which host and microbiota produce the same metabolite, are particularly challenging to quantify and to predict.

The gut microbiome also impacts intravenously administered and rapidly absorbed compounds due to biliary excretion, and adverse reactions to microbiome-derived drug metabolites have caused human fatalities. Gut microbes collectively encode 150-fold more genes than the human genome, including a rich repository of enzymes with the potential to metabolize drugs and hence influence their pharmacology. Gut microbes can impact drugs inside and outside the gut. The gut microbiota is implicated in the metabolism of many medical drugs, which has important consequences for efficacy, toxicity, and interpersonal variation in drug response. Anecdotal examples exist of drug metabolism being influenced by gut microbes.

However, no rules exist to identify microbiome-targeted drugs or drug-targeting microbes. As a result, we cannot predict how an individual's gut microbial community will influence the efficacy and safety of almost any drug. The existing technology focus on understanding how human genes impact drug metabolism. There are no previous inventions that allow systematic assessment of whether and how a drug candidate will be metabolized by gut microbiota.

### SUMMARY

As specified above, there is a need in the art for disentangling host and microbial contributions to drug metabolism. It is desirable to gain a quantitative understanding of these host and microbiome-encoded metabolic activities in order to clarify how nutritional, environmental, genetic and galenic factors impact drug metabolism and enable tailored intervention strategies to improve drug responses. It is also desirable to identify optimal drug candidates earlier in the development process so as to reduce enormous cost and time used for drug development.

The present invention addresses these and other needs by providing high-throughput methods and databases for a) predicting whether and how a drug candidate will be metabolized by microbiota (e.g., for characterizing the drug candidate's efficacy and toxicity and comparing to other candidates); b) predicting how inter-individual microbiota variations impact how a drug is metabolized (e.g., for predicting toxicity and/or efficacy and/or pharmacokinetics of the drug for an individual patient, including selecting the most appropriate drug for a given patient and selecting an effective dose and/or regimen of the drug); c) identifying drug-metabolizing microbiota taxa (e.g., for predicting drug toxicity and/or efficacy, or for altering microbiota to achieve the lowest toxicity and highest efficacy for a given drug); d) identifying microbial genes that confer specific drug metabolizing capabilities (e.g., for predicting drug toxicity and/or efficacy, or for altering microbiota to achieve the lowest toxicity and highest efficacy for a given drug); e) predicting host and microbiota contributions to blood/systemic drug and metabolite levels; f) identifying a subpopulation of patients for the drug administration and/or stratification in clinical trials, and g) analyzing drug-drug interactions (e.g., for developing new co-administration regimens for lowering toxicity and/or increasing efficacy of a given drug). In one aspect, the disclosed technology relates to a system that includes a non-transitory storage medium and a processor. The non-transitory storage medium stores information of levels of parent drug and drug metabolites for a plurality of oral drugs, by each of a plurality of microbiotas and by each of a plurality of microbes in pure culture. The processor is in communication with the non-transitory storage medium. The processor is configured to execute a predictor module which implements a computational model to perform the following: quantitatively disentangling host and microbiota contributions to drug metabolism; predicting how a subject's microbiota will metabolize a drug candidate; predicting how the metabolization impacts the drug candidate and metabolite exposure in circulation; and predicting whether the drug candidate will be metabolized by the microbiota.

Another aspect of the disclosed technology relates to a system that includes a non-transitory storage medium and a processor. The non-transitory storage medium stores information of levels of parent drug and drug metabolites for a plurality of oral drugs, by each of a plurality of microbiotas and by each of a plurality of defined and characterized (e.g. genome-sequenced) microbes in pure culture. The processor is in communication with the non-transitory storage medium. The processor is configured to execute a predictor module which implements a computational model to perform the following: receiving a microbiome composition as input; and generating an output that predicts kinetics of microbiome-meditated metabolism of a drug candidate.

An additional aspect of the disclosed technology relates to a system that includes a non-transitory storage medium and a processor. The non-transitory storage medium stores information of levels of parent drug and drug metabolites for a plurality of oral drugs, by each of a plurality of microbiotas and by each of a plurality of defined and characterized (e.g. genome-sequenced) microbes in pure culture. The processor is in communication with the non-transitory storage medium. The processor is configured to execute a predictor module which implements a computational model to perform the following: receiving a chemical structure of a drug candidate as input; and predicting as output whether the drug candidate will be metabolized by each of the plurality of microbiotas and the microbes in the non-transitory storage medium.

Another aspect of the disclosed technology relates to a system that includes a non-transitory storage medium and a processor. The non-transitory storage medium stores information of levels of parent drug and drug metabolites for a plurality of oral drugs, by each of a plurality of microbiotas and by each of a plurality of genome-sequenced microbes in pure culture. The processor is in communication with the non-transitory storage medium. The processor is configured to execute a predictor module which implements a pharmacokinetic model to perform the following: predicting microbiome contribution to drug and metabolite exposure over time.

An additional aspect of the disclosed technology relates to a method that includes storing, by a non-transitory storage medium, information of levels of parent drug and drug metabolites for a plurality of oral drugs, by each of a plurality of microbiotas and by each of a plurality of defined and characterized (e.g. genome-sequenced) microbes in pure culture. The method also includes executing, by a processor in communication with the non-transitory storage medium a predictor module which implements a computational model to perform the following: quantitatively disentangling host and microbiome contributions to drug metabolism; predicting how a subject's microbiome will metabolize a drug candidate; predicting how the metabolization impacts the drug candidate and metabolite exposure in circulation; and predicting whether the drug candidate will be metabolized by a microbiota.

Another aspect of the disclosed technology relates to a method that includes storing, by a non-transitory storage medium, information of levels of parent drug and drug metabolites for a plurality of oral drugs, by each of a plurality of microbiotas and by each of a plurality of defined and characterized (e.g. genome-sequenced) microbes in pure culture. The method also includes executing, by a processor in communication with the non-transitory storage medium, a predictor module which implements a pharmacokinetic model to perform the following: receiving a microbiome composition as input; and generating an output that predicts kinetics of microbiota-meditated metabolism of a drug candidate.

An additional aspect of the disclosed technology relates to a method that includes storing, by a non-transitory storage medium, information of levels of parent drug and drug metabolites for a plurality of oral drugs, by each of a plurality of microbiotas and by each of a plurality of genome-sequenced microbes in pure culture. The method also includes executing, by a processor in communication with the non-transitory storage medium, a predictor module which implements a computational model to perform the following: receiving a chemical structure of a drug candidate as input; and predicting as output whether the drug candidate will be metabolized by each of the plurality of microbiotas and the microbes in the non-transitory storage medium.

Another aspect of the disclosed technology relates to a method that includes storing, by a non-transitory storage medium, information of levels of parent drug and drug metabolites for a plurality of oral drugs, by each of a plurality of microbiotas and by each of a plurality of defined and characterized (e.g. genome-sequenced) microbes in pure culture. The method also includes executing, by a processor in communication with the non-transitory storage medium, a predictor module which implements a computational model to perform the following: predicting microbiome contribution to drug and metabolite exposure over time.

Various aspects of the described illustrative embodiments may be combined with aspects of certain other embodiments to realize yet further combinations. It is to be understood that one or more features of any one illustration may be combined with one or more features of the other arrangements disclosed.

### BRIEF DESCRIPTION OF THE DRAWINGS

FIG. 1 illustrates a block diagram of an overall system according to one aspect of the disclosed technology.

FIGS. 2A-C are flow charts illustrating steps performed by the system according to one aspect of the disclosed technology.

FIG. 3 illustrates commensal-mediated drug metabolism clusters according to bacterial phylogeny and drug chemical structure with examples.

FIG. 4A illustrates schematic representation of host and microbiome processes that contribute to drug metabolism in connection with BRV to BVU conversion by mammalian and microbial enzymes in vivo.

FIG. 4B illustrates chemical structure of BRV and BVU.

FIG. 4C illustrates BRV and BVU concentration in the different intestinal segments over time, where each field represents the mean value of five animals.

FIG. 4D illustrates BRV and BVU serum kinetics in CV and GF mice.

FIG. 4E illustrates BRV and BVU liver concentrations in CV and GF mice.

FIG. 5A illustrates a PCoA plot of the chemical diversity of 1,760 FDA-approved drugs based on molecular fingerprints as part of systematic analysis of microbiome-mediated drug metabolism by combinatorial pooling.

FIG. 5B illustrates combinatorial pooling of selected 271 drugs into 21 pools (A-U).

FIG. 5C illustrates time-course results of 11 random individuals' metabolism of sulfasalazine and risperidone, two drugs known to be metabolized by the gut microbiota in vivo.

FIG. 5D illustrates time-course results of 11 random individuals' metabolism of famciclovir and tranilast, two drugs not previously associated with microbiome-mediated metabolism.

FIGS. 6A-E illustrates the identification of drug-metabolizing bacterial species in a bacterial community using deflazacort as an example, where deflazacort metabolism by 11 human donor microbiomes linear regression analysis to relate genus and species abundance to deflazacort metabolism rate.

FIG. 7 Distribution of functional chemical groups in drugs that are metabolized or not metabolized across the 76 tested bacterial strains. Abundance of each chemical group among the 271 selected drugs and 2099 clinical drugs (from DrugBank) is indicated.

FIG. 8 illustrates the phyla-based distribution of the 76 human gut commensals that were tested for the metabolism of 271 drugs.

FIG. 9A illustrates the log₁₀ fold difference in abundance of each compound (drugs and metabolites represented as dots) in bisacodyl-containing pools versus others at T₁₂ₕ plotted against statistical significance for C. asparaginoforme. Compounds with significant enrichment in bisacodyl-containing pools are shown in black with observed mass noted.

FIG. 9B illustrates the log₁₀ fold change at T₁₂ₕ v. T₀ₕ for drugs and their specific metabolites when metabolized by C. asparaginoforme plotted against statistical significance. Compounds that change over time are shown in black, others in gray.

FIGS. 10A-B illustrate loss-of-function strategy for identifying microbiome-encoded DMEs using a library of transposon mutants representing disruption in 70% of B. thetaiotaomicron (Bt) non-essential genes as shown by the log 2 fold change plot for each mutant and the time course plot of the unmarked non-polar deletion of bt4554, respectively.

FIG. 10C illustrates the necessity of BT0152 to metabolize roxatidine acetate as shown in the log 2 fold change plot for Δbt0152 and E. coli (pbt0152).

FIGS. 11A-G illustrates gain-of-function strategy for identifying microbiome-encoded DMEs as shown by screening pools of E. coli clones, each expressing a B. thetaiotaomicron (Bt) genomic fragment with Bt-metabolized drugs to identify pools with activity against diltiazem.

FIG. 12A illustrates enzymatic conversion of BRV to BVU by human and murine S9 liver fractions.

FIG. 12B illustrates in vitro conversion of BRV to BVU by human and murine gut microbial communities.

FIG. 12C illustrates BRV and BVU serum kinetics in CV and GF mice.

FIG. 12D illustrates cecal BRV and BVU concentrations over time in CV and GF mice.

FIG. 12E illustrates total amount of BRV and BVU over time in the cecum and feces of CV and GF mice.

FIG. 12F illustrates liver concentrations of BRV and BVU over time in CV and GF mice.

FIG. 12G illustrates liver thymine concentrations over time in CV and GF mice.

FIG. 13 illustrates BRV and BVU kinetics in intestinal compartments of CV and GF mice.

FIG. 14A illustrates BRV conversion to BVU by representative human gut isolates.

FIG. 14B illustrates log₂ fold change of BRV and BVU concentrations of B. thetaiotaomicron transposon insertion mutants (dark grey, n=1290) compared to media controls (light grey, n=83) after 24 h of incubation.

FIG. 14C illustrates BRV conversion by B. thetaiotaomicron wildtype (n=4), bt4554 mutant (n=4), and complemented strains expressing bt4554 at different levels (n=8).

FIG. 14D illustrates serum BRV and BVU kinetics in GN^{WT} and GN^{MUT} mice.

FIG. 14E illustrates liver BRV and BVU kinetics in GN^{WT} and GN^{MUT} mice.

FIG. 14F illustrates liver thymine concentrations over time in GN^{WT} and GN^{MUT} mice.

FIG. 14G illustrates intestinal BRV and BVU concentrations over time in GN^{WT} and GN^{MUT} mice.

FIG. 14H illustrates cecal and fecal BRV and BVU concentrations over time in individual GN^{WT} and GN^{MUT} animals.

FIG. 15A illustrates distribution of transposon insertion relative position within each gene in the original and condensed library.

FIG. 15B illustrates number of insertions per strain in the original and condensed library.

FIG. 15C illustrates predicted number of genes perturbed by transposon insertions per strain in the original and condensed library after selection of mutants that maximize predicted polar effects on downstream genes in the same operon.

FIG. 16 illustrates BRV to BVU conversion by B. thetaiotaomicron VPI-5482 parental and the tdk (WT) strain used as a genetic background for bt4554 deletion and complementation.

FIG. 17A illustrates fecal colony-forming unit (CFU) counts of GN^{WT} and GN^{MUT} mice 4 days after colonization (n>20).

FIG. 17B illustrates kinetics of anaerobic growth of B. thetaiotaomicron wildtype and bt4554 strains in GMM.

FIG. 18A illustrates comparison of BRV and BVU serum kinetics between GF and GN^{MUT} mice.

FIG. 18B illustrates BRV and BVU kinetics in intestinal compartments of GN^{WT} and GN^{MUT} mice following oral BRV gavage.

FIG. 19A illustrates schematic representation of compartments and sub-processes included in a physiologically based model of host and microbial contribution to BRV, SRV, and BVU pharmacokinetics.

FIG. 19B illustrates parameterization of microbiota-independent processes using measurements from GN^{MUT} mice.

FIGS. 19C-E illustrate parameterization of microbiota-dependent intestinal drug metabolism and prediction of microbial and host contributions to serum BVU in GN^{WT} CV mice.

FIG. 19F illustrates absolute metabolite exposure and relative bacterial contribution to serum BVU as a function of host and microbial drug metabolism rate at a given bioavailability.

FIG. 19G illustrates predicting host and microbial contribution to serum BVU after oral SRV administration to CV mice.

FIG. 20A illustrates fitting and prediction of BRV and BVU kinetics in different compartments of GN^{WT} mice following oral BRV administration.

FIG. 20B illustrates fitting and prediction of BRV and BVU kinetics in different compartments of CV mice after oral BRV administration.

FIG. 20C illustrates normalized sensitivity analysis of the fully parameterized pharmacokinetic model for total BVU serum levels, and relative BVU serum contributions by the host and microbes.

FIGS. 21A-F illustrate simulation of absolute metabolite exposure and relative microbial contribution to serum metabolite exposure as a function of host and microbiome metabolic capacity and different drug bioavailabilities.

FIGS. 21G-L illustrate simulation of absolute metabolite exposure as a function of host and microbiome metabolic capacity and different drug bioavailabilities.

FIG. 22A illustrates chemical structure of SRV and BVU.

FIG. 22B illustrates that that SRV is slowly converted to BVU by human and murine S9 liver fractions.

FIG. 22C illustrates that B. thetaiotaomicron slowly converts SRV to BVU in a BT4554-dependent manner.

FIGS. 23A-B illustrate SRV and BVU serum and liver kinetics and thymine liver kinetics in CV and GF mice following oral SRV administration.

FIG. 23C illustrates intestinal compartment concentrations of SRV and BVU over time.

FIG. 23D illustrates SRV and BVU kinetics in intestinal compartments of individual CV and GF mice.

FIG. 23E illustrates parameterization of microbiota-independent SRV kinetics using measurements from GF mice.

FIG. 23F illustrates predicting microbial and host contributions to serum BVU in CV mice after SRV administration.

FIG. 24 illustrates verified gene-drug-metabolite interactions.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and/or circuitry have been described at a relatively high-level, without detail, in order to avoid unnecessarily obscuring aspects of the present teachings.

### 1. Overall System

The present application relates to a system 100 that may predict how a person's microbiome will metabolize a drug, and how the metabolization will impact drug and metabolite exposure in circulation and other tissues. The system 100 may predict whether a drug candidate will be metabolized by the microbiota, what drug metabolites will be produced, and how microbiome variation will impact these events. The system 100 may forecast variation in drug response at a broader scale, including large metagenomic datasets and comprehensive chemical libraries of drug candidates or new chemical entities.

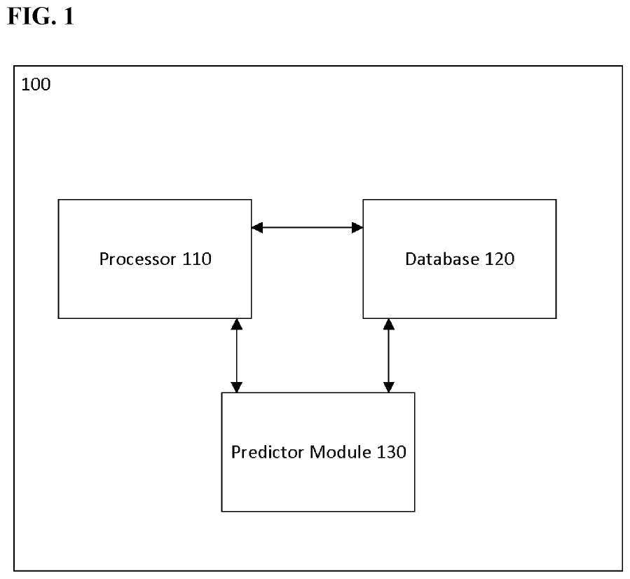

As illustrated in FIG. 1, the system 100 may include a processor 110, a database 120 and a predictor module 130 executable by the processor 110.

The database 120 may be a non-transitory storage medium. The database 120 may store information of levels of parent drug and drug metabolites for each of approved oral drugs, by each of a plurality of microbiomes and by each of a plurality of defined bacterial species in pure culture.

In one example, the database 120 may store information of levels of parent drug and drug metabolites for each of 271 approved oral drugs, by each of 60 human gut microbiomes from unrelated human donors and by each of 76 defined bacterial species/strains in pure culture. This represents 36,314 time-series data-sets. The human donors and human microbiomes may be selected to maximize representation of diverse bacterial species.

In another example, the database 120 may store measurements of drug and metabolite levels, collected over time and across tissues of subjects. The subjects may be humans or mice carrying (genetically manipulated) gut microbes.

In one example, the database 120 may store information of tens of thousands of drug-microbiome interactions. Such information may reveal how bacteria metabolize medical drugs.

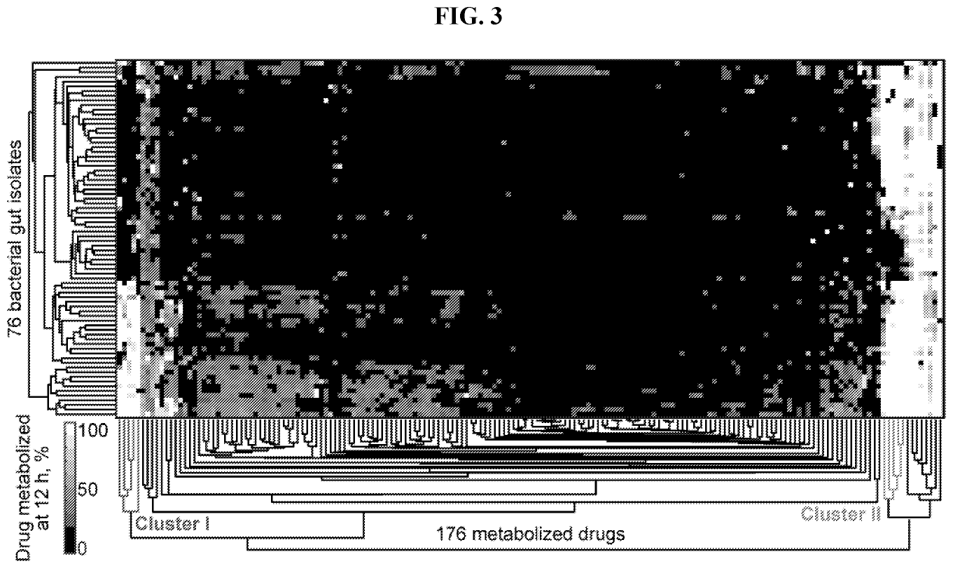

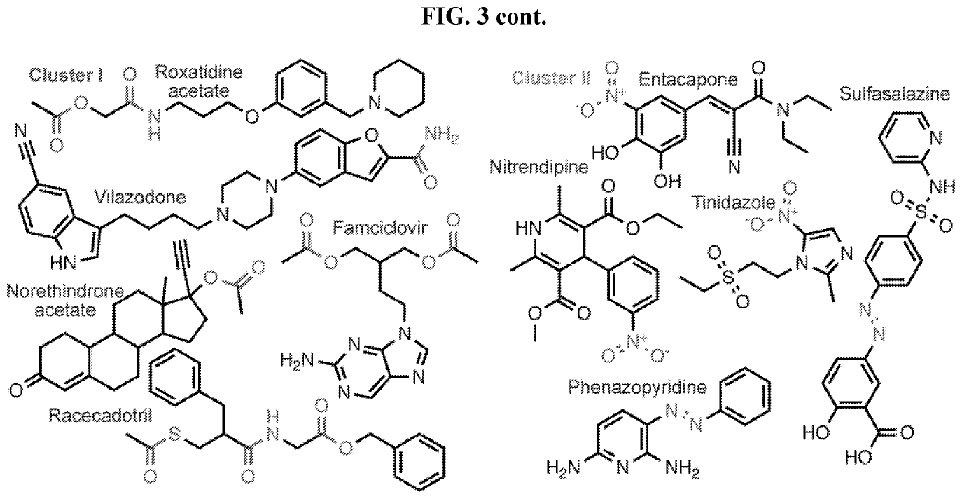

The database 120 may store information of hierarchical clustering or other distance measurements of a set of commensals based on their ability to metabolize drugs, where related bacteria are clustered together at broad (phyla) and specific (strain) levels. For example, the database 120 may store information of hierarchical clustering of 76 commensals based on their ability to metabolize 271 drugs. As depicted in FIG. 3, hierarchical clustering, based only on drug metabolism capacity, may cluster related species from phylum to strain level, and may also cluster structurally similar drugs.

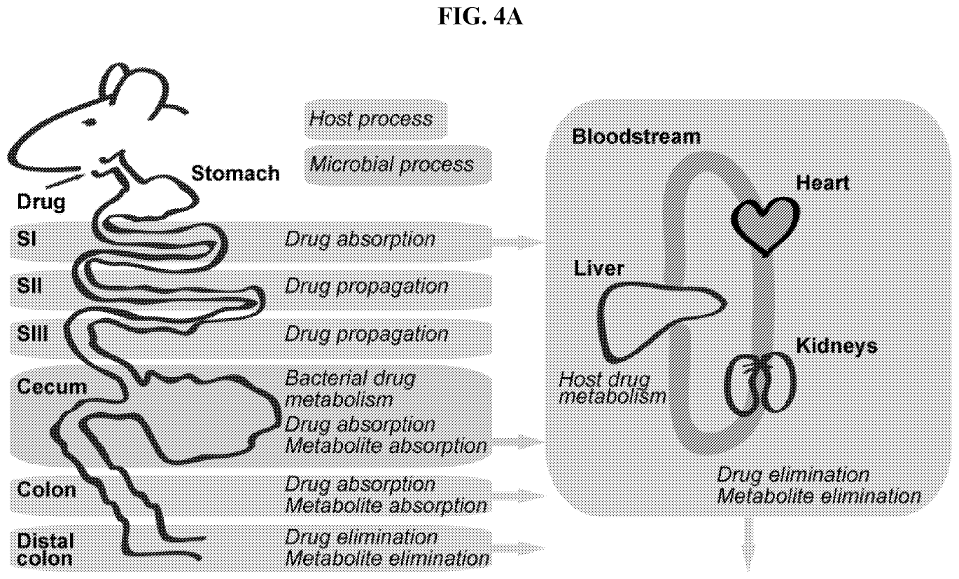

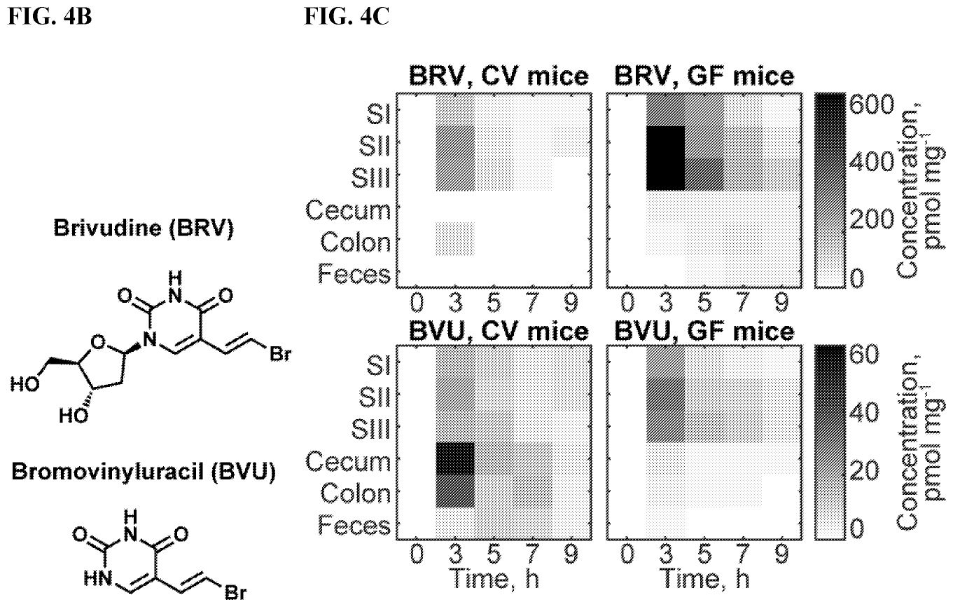

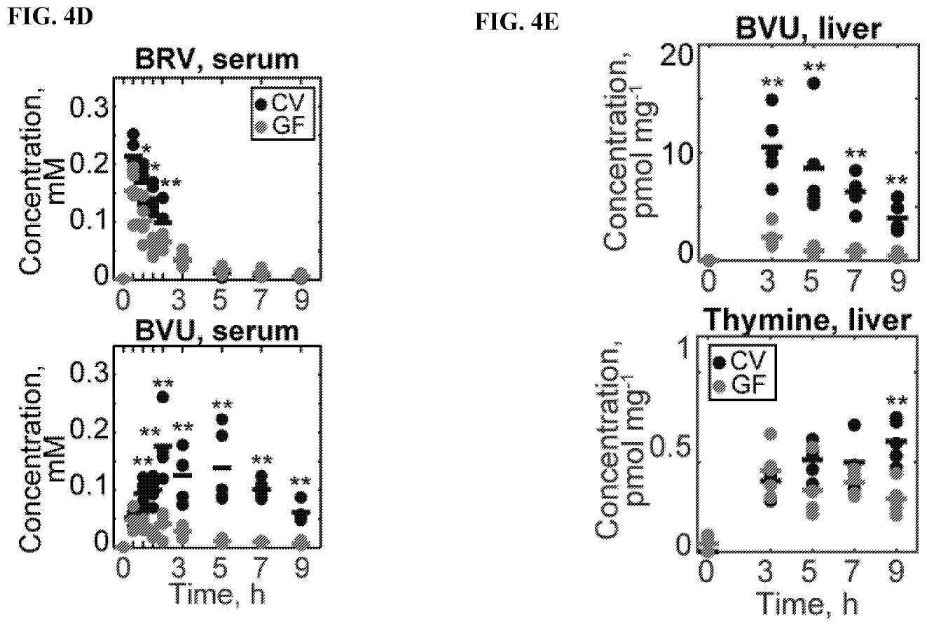

With reference to FIG. 4A, the processor 110 may execute the predictor module 130 which implements a pharmacokinetic model that quantitatively disentangles host and microbiome contributions to drug metabolism. The prediction module 130 may be implemented using the MatLab 2017b SimBiology Toolbox (MathWorks). The processor 110 by executing the predictor module 130 may forecast variation in drug response at a broader scale, including large metagenomic datasets and comprehensive chemical libraries of drug candidates or new chemical entities. The processor 110 by executing the predictor module 130 may predict how a person's microbiome will metabolize a drug, and how these activities will impact drug and metabolite exposure in circulation. The processor 110 may predict whether a drug candidate will be metabolized by the microbiota, what drug metabolites will be produced, and how microbiome variation will impact these events. The processor 110 by executing the predictor module 130 may perform processes on new drugs, drug candidates, and other molecules.

The predictor module 130 may be based on machine learning algorithms.

In one example, the predictor module 130 may implement a random forest algorithm as discussed in Goodman A L, McNulty N P, Zhao Y, Leip D, Mitra R D, Lozupone C A, Knight R, Gordon J I. Identifying genetic determinants needed to establish a human gut symbiont in its habitat. Cell Host Microbe. 2009; 6(3):279-89. Epub 2009/09/15. doi: S1931-3128(09)00281-9 [pii] (hereinafter, “Goodman 2009”), the entire content which is incorporated by reference herein.

The predictor module 130 may be optimized by experimental testing of thousands of compounds through combinatorial pooling and the use of different liquid and solid chromatographic phases together with other parameters to maximize the number of detected compounds and derived metabolites.

The processor 110 by executing the predictor module 130 may predict the kinetics of microbiome-mediated metabolism of a drug as output after receiving any microbiome composition as input. The processor 110 may directly measure the kinetic constants of drug metabolism of many drugs and drug candidates by dozens to hundreds of individual microbial communities in parallel. The processor 110 may perform a high-throughput process for experimentally measuring whether and how many drug candidates (hundreds or thousands) are metabolized by the microbiota.

The processor 110 by executing the predictor module 130 may predict whether and how a drug will be metabolized by a microbiome composition as output after receiving the microbiome composition as input. The processor 110 may predict how inter-individual microbiota variations will impact how a drug is metabolized, such as for predicting toxicity and/or efficacy and/or pharmacokinetics of the drug for an individual patient, including selecting the most appropriate drug for a given patient and selecting an effective dose and/or regimen of the drug. The processor 110 may predict whether and how a drug candidate will be metabolized by the microbiota, such as for characterizing the drug candidate's efficacy and toxicity and comparing to other candidates. The processor 110 may identify drug-metabolizing microbiota taxa, such as for predicting drug toxicity and/or efficacy, or for altering microbiota to achieve the lowest toxicity and highest efficacy for a given drug. The processor 110 may identify microbial genes that confer specific drug metabolizing capabilities, such as for predicting drug toxicity and/or efficacy, or for altering microbiota to achieve the lowest toxicity and highest efficacy for a given drug. The processor 110 may identify individual genes in the microbiome that determine systemic levels of a toxic drug metabolite, and then determine the toxicity of a given drug.

The microbiome composition input may be defined by 16S-RNA sequencing, metagenomics, or other methods. In one example, no information about chemical structure of the drug needs to be provided to the processor 110.

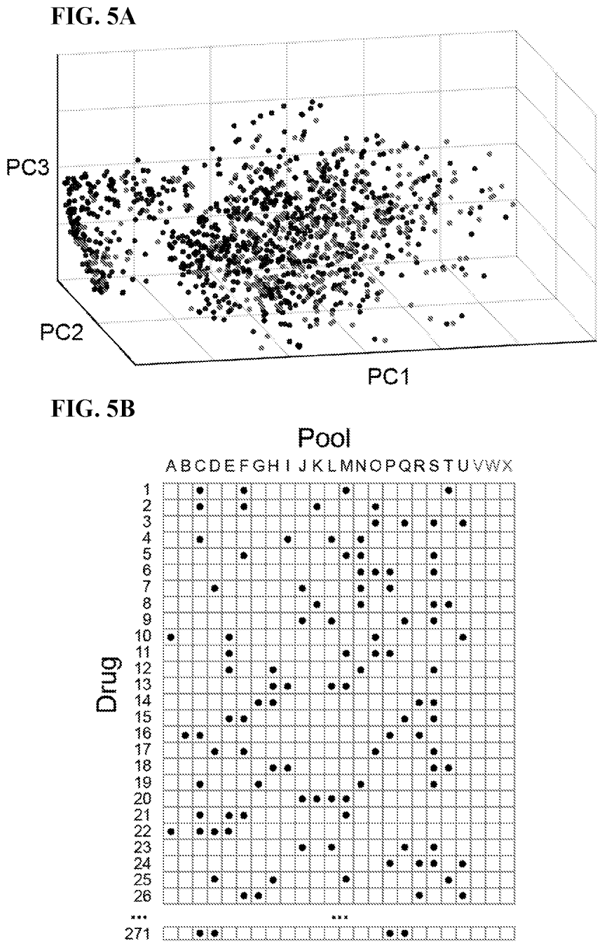

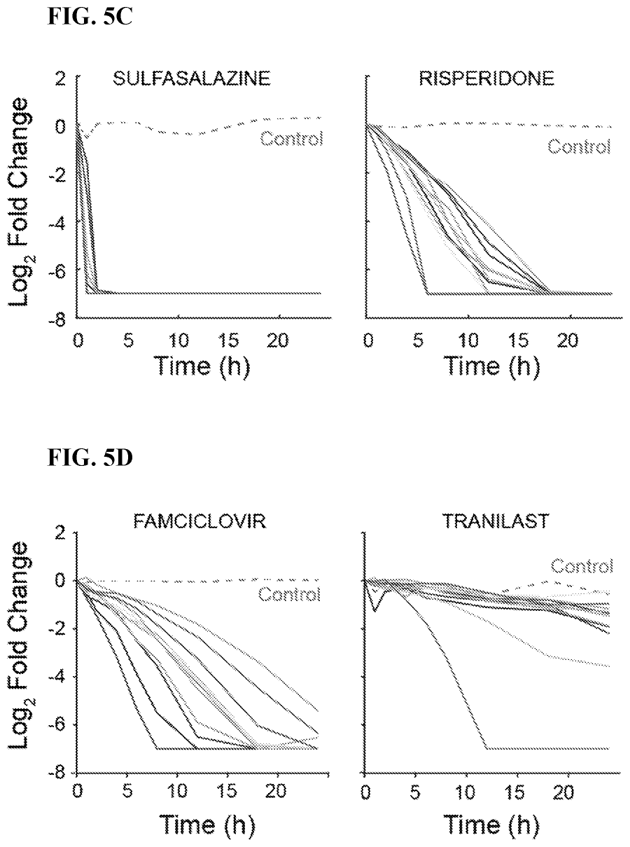

For example, the processor 110 may execute the predictor module 130 to receive microbiota composition and compound chemical fingerprint as input to estimate the kinetic coefficient of metabolism for the drug by that community or species as output. Experimental data may be used to train and validate the predictor module 130. Such experimental data may include the already measured kinetic coefficients of drug metabolism from preliminary data. In one example, preliminary data may include information of 271 drugs by 60 communities as shown in FIG. 5A, and by 76 species/strains as shown in FIG. 3. Experimental data may also include phylogenetic and gene content data from metagenomes and genomes. Genomes may be already sequenced, and 16S data may be already available for the 60 communities. Input data may also include chemical fingerprints for different compounds, such as 271 drugs. Parameters of the predictor module 130 may be fit by minimizing prediction error, such as minimizing percent-out-of-bag error and evaluated by ten-fold cross-validation.

In one example, the processor 110 may predict the kinetics of metabolism of 271 drugs by any microbiome, and whether a new drug will be targeted by any of 60 test microbiomes.

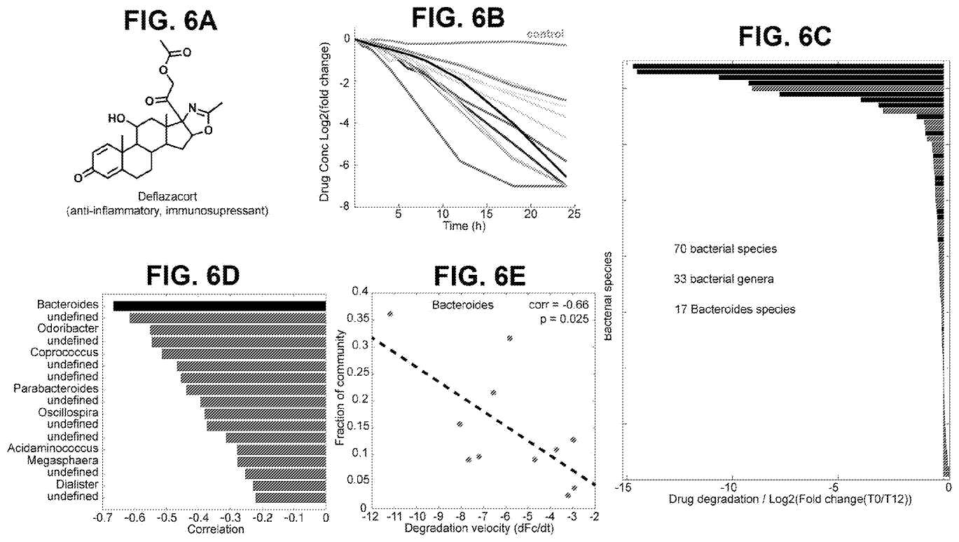

With reference FIGS. 5C-D, because many drugs exhibit donor-dependent metabolism kinetics, linear regression analysis may be conducted on compositional data, such as V4-targeted 16S sequences, and drug metabolism kinetics from an initial set of donors, e.g., 11 donors. In FIGS. 5C and D, each line represents one of 11 individual human donors randomly selected from 60 studied. The dashed line shows time-course results from no-microbiota control incubations. Even with this limited taxonomic resolution and sample count, the processor 110 by executing the predictor module 130 may successfully identify significant correlations between drug metabolism kinetics and microbiome community composition as illustrated in FIG. 6A-D) that are independently recapitulated by single-species studies as illustrated in FIG. 6E. As shown in FIG. 6E, quantification of deflazacort metabolism by individual species shows that most of the fast metabolizers of this drug are Bacteroides (black).

The processor 110 by executing the predictor module 130 may predict as output whether a drug will be metabolized by each of microbial communities and species in the database 120 after receiving any drug structure as input. For example, by relying on information stored in the database 120, the processor 110 may take the chemical structure of a drug candidate as input and predict whether and how a drug candidate will be metabolized by the microbiota as output.

With reference to FIG. 3, the processor 110 by executing the predictor module 130 may perform hierarchical clustering analysis to place chemically related drugs together based simply on their tendency to be metabolized by the same set of species.

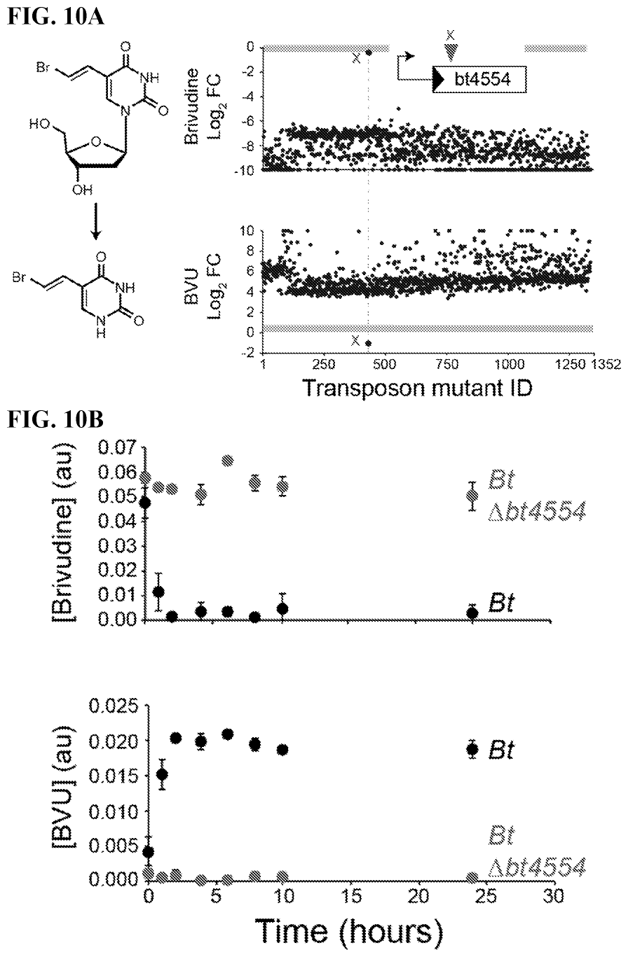

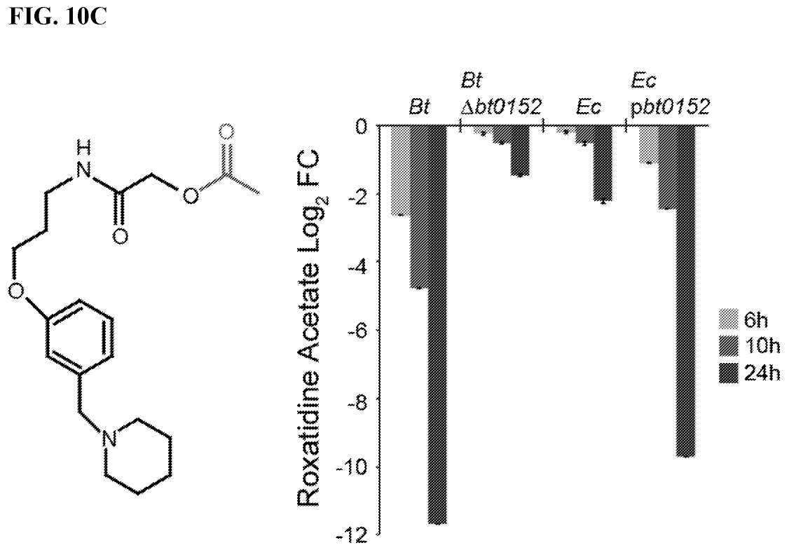

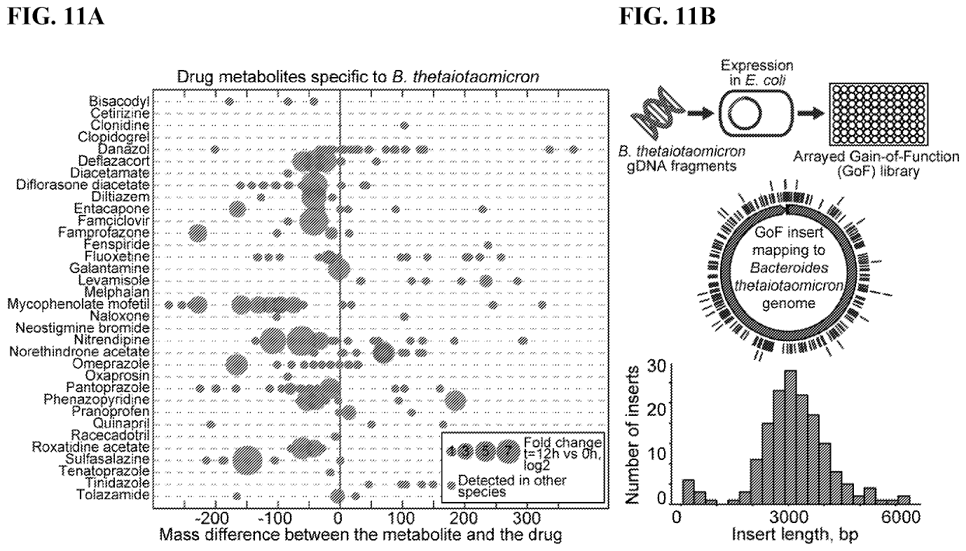

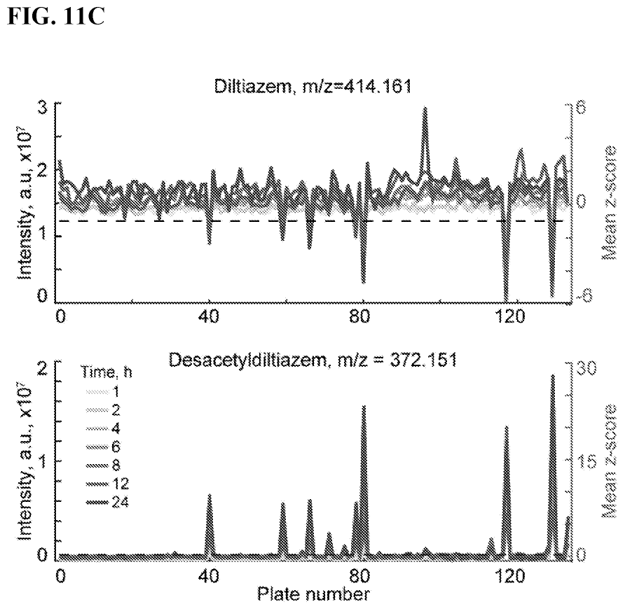

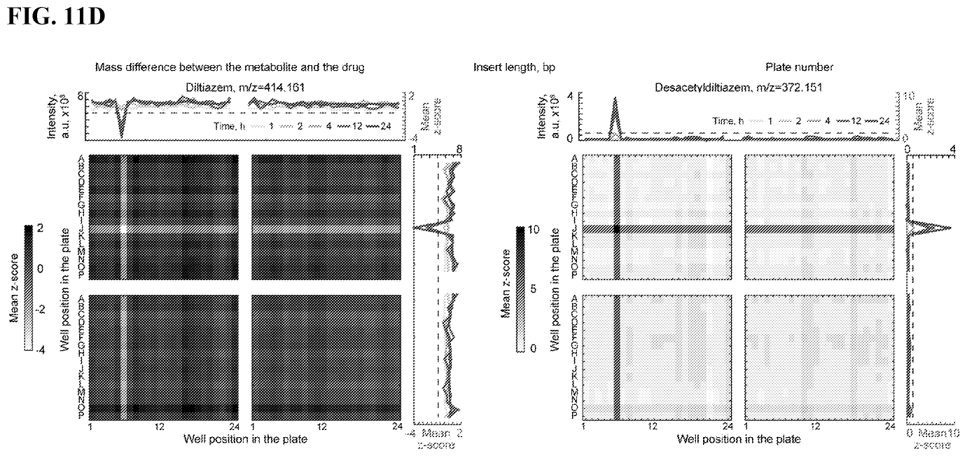

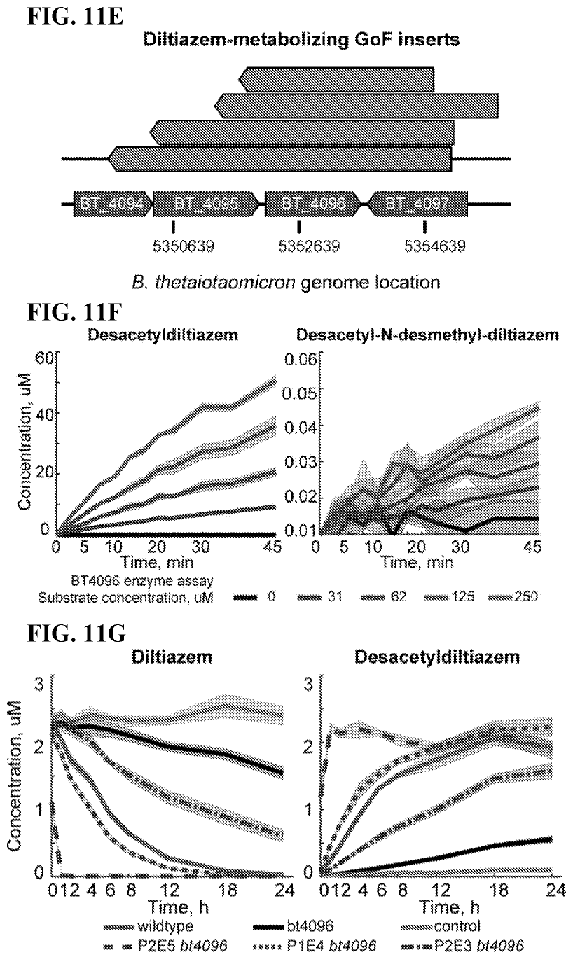

In one example, the processor 110 by executing the predictor module 130 may predict microbiome contribution to drug and metabolite exposure over time. For example, this approach predicts host and microbiome contribution to drug/metabolite exposure separately in the following steps. First, to predict host contribution, studies in germfree mice are conducted. Alternatively, studies in mice colonized with bacterial species or mutants that lack drug metabolizing activity are used. Mutants lacking drug metabolizing activity are determined using the gain-of-function and loss-of-function approaches described in connection with FIGS. 10A-B and FIG. 11 for identifying microbiome-encoded DMEs, and then deleting these genes from the relevant bacterial genome. Second, to predict microbiome contribution, drug and metabolite levels are measured over time along the length of the gut in mice that do carry drug-metabolizing bacteria (complete microbiome communities, or single species with the relevant DMEs present). The levels of drug and metabolite absorbed into circulation are predicted from these gut measurements. Third, the measurements of the first above two steps are used to parameterize equations that use mass action kinetics to fit rates to host and microbiome contribution to drug and metabolite levels in serum over time. Fourth, these parameterized equations can be combined to predict total drug and metabolite levels over time.

With reference to FIG. 4A, the processor may combine host-specific processes with microbiota-specific processes to provide general insight into how these processes influence the contribution of the microbiome to systemic drug and metabolite exposure. Host-specific processes may include drug absorption and elimination, oral bioavailability, host metabolism and metabolite elimination. Microbiota-specific processes may include intestinal transit, microbial metabolism, and metabolite absorption from the large intestine. In one example, the processor 110 by executing the predictor module 130 may quantitatively predict the contribution of the gut microbiome to systemic drug and metabolite exposure, as a function of bioavailability, host and microbial drug metabolizing activity, drug and metabolite absorption, and intestinal transit kinetics.

The processor 110 by executing the predictor module 130 may identify drug-drug interactions. The processor 110 may identify novel drug metabolites produced by microbiome activity that can form the basis of drug-drug interaction, such as for developing new co-administration regimens for lowering toxicity and/or increasing efficacy of a given drug.

This approach may apply to human-associated microbial communities from other body habitats, non-human microbial communities including those from preclinical animal models and others, and to non-drug xenobiotics and non-xenobiotic molecules.

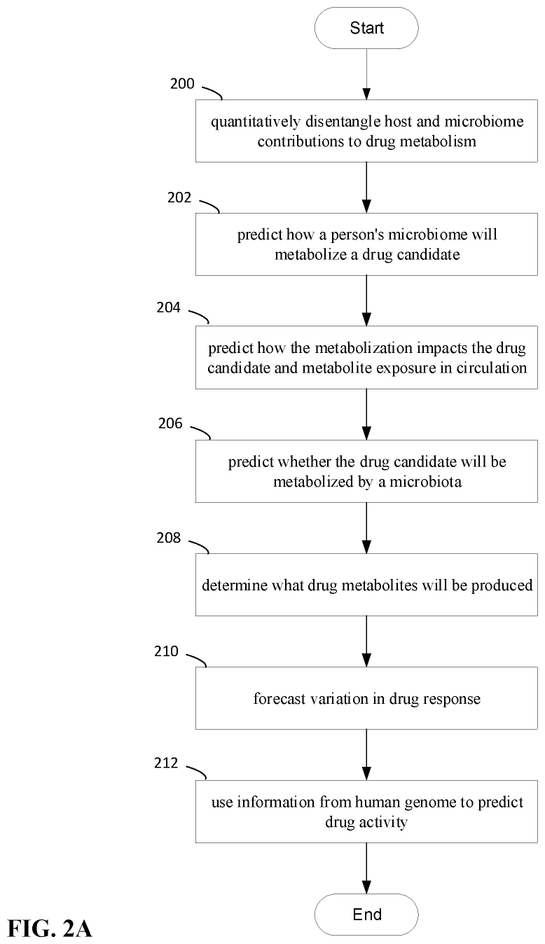

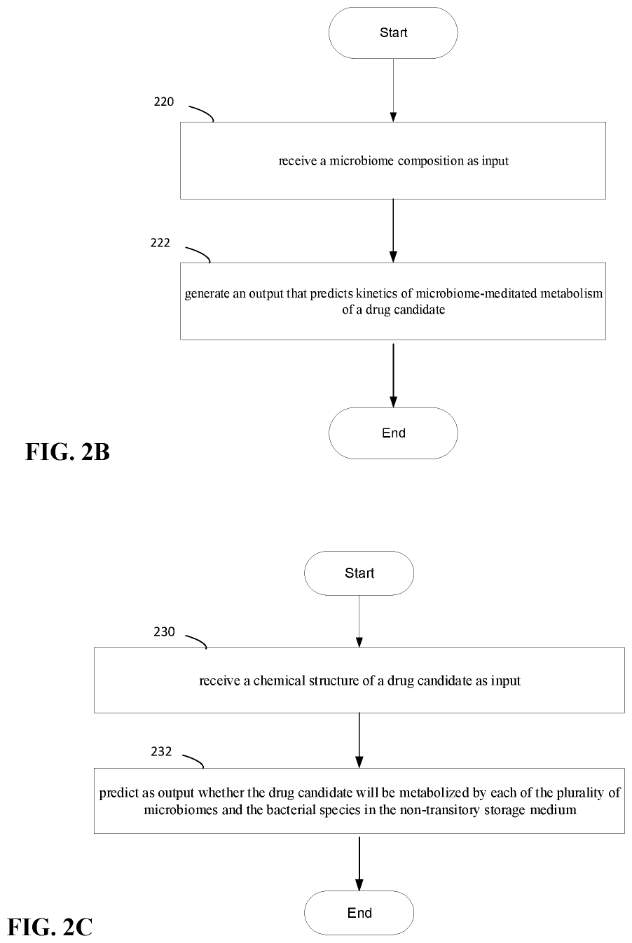

FIGS. 2A-C are flow charts illustrating example steps that may be executed by the processor 110.

With reference to FIG. 2A, at 200, the processor 110 may quantitatively disentangle host and microbiome contributions to drug metabolism through three steps, as demonstrated for Brivudine and Sorivudine described later. First, inputs include levels of drug and metabolite(s), over time, along the length of the GI tract, in serum, in liver, in urine, and in other tissues. These levels may be measured in germfree animals, antibiotic-treated conventional animals, gnotobiotic animals carrying a defined microbiome, gnotobiotic animals carrying a complete mouse, human, or other microbiome, or conventional animals. These levels may also be measured in gnotobiotic mice carrying genetically manipulated microbes whose expression of drug-metabolizing enzyme(s) are zero, over-expressed, or any range of expression in between. Second, these inputs are fed into a physiology based pharmacokinetic model that defines rates for transitions of drug through different body sites, of metabolite through different body sites, and of conversion from drug to metabolite at different body sites (including by the gut microbiota or other microbiota). For example, the contribution of the microbiome to serum metabolite levels are estimated from the levels of drug in the gut of mice lacking the microbiome-encoded drug metabolizing activity. The reduction in GI drug levels in mice carrying the microbiome-encoded drug metabolizing activity indicates the conversion of drug to metabolite by the microbiota. The difference between the levels of metabolite observed in the GI tract and the levels expected from conversion of the known concentration of drug is used to estimate the levels of metabolite that are absorbed into circulation from the gut. Examples of other parameters and how they are estimated is shown in the example for Brivudine and Sorivudine described later. Third, these parameters are used to provide as output 1) predicted levels of serum (or other tissue) drug and metabolite exposure over time that result from both host and microbiome activities, 2) relative contribution of host and microbiome to these serum (or other tissue) levels over time, 3) impact of varying any of the parameters included in the model on any of these predictions. For example, this approach reveals how individual or parallel variation of host and microbiome drug metabolizing activity and drug bioavailability impacts the total serum exposure of drug and metabolites and the host vs. microbiome contribution to these total serum exposures.

At 202, the processor 110 may predict how a person's microbiome will metabolize a drug candidate. As described above, four steps may be taken. First, to predict host contribution, studies in germfree mice are conducted. Alternatively, studies in mice colonized with bacterial species or mutants that lack drug metabolizing activity are used. Mutants lacking drug metabolizing activity are determined using the gain-of-function and loss-of-function approaches described in connection with FIGS. 10A-B and FIG. 11 for identifying microbiome-encoded DMEs, and then deleting these genes from the relevant bacterial genome. Second, to predict microbiome contribution, drug and metabolite levels are measured over time along the length of the gut in mice that do carry drug-metabolizing bacteria (complete microbiome communities, or single species with the relevant DMEs present). The levels of drug and metabolite absorbed into circulation are predicted from these gut measurements. Third, the measurements of the first above two steps are used to parameterize equations that use mass action kinetics to fit rates to host and microbiome contribution to drug and metabolite levels in serum over time. Fourth, these parameterized equations can be combined to predict total drug and metabolite levels over time.

At 204, the processor 110 may predict how the metabolization impacts the drug candidate and metabolite exposure in circulation. Three steps may be taken. First, structures of drug metabolites are predicted from the combinatorial screening and constraints of the parent drug structure. Second, the drug-likeness of these predicted drug metabolites are estimated from these structures using standard methods, including Lipinksi's Rule of Five. Third, bioavailability and enterohepatic circulation of drugs and metabolites are measured using standard methods, such as assessment of serum exposure from oral vs. IV administration. These elements feed a likelihood score for microbiome impact of drug and metabolite levels in circulation.

At 206, the processor 110 may predict whether the drug candidate will be metabolized by a microbiota. The drug candidate may be represented as chemical fingerprint. These chemical fingerprints may be used by a machine learning algorithm trained on the reference drugs to predict the kinetics of drug transformation.

At 208, the processor 110 may determine what drug metabolites will be produced. The drug candidate may be represented in a chemical fingerprint. These chemical fingerprints may be used by a machine learning algorithm trained on the reference drugs to predict the metabolic transformation performed by the microbiota.

At 210, the processor 110 may forecast variation in drug response. This may be done if the metabolites generated by the microbiota impact drug response.

At 212, the processor 110 may use information from human genome to predict drug activity. The processor 110 may use available data from literature or unpublished studies as the source of this information.

With reference to FIG. 2B, at 220, the processor 110 may receive a microbiome composition as input. At 222, the processor 110 may generate an output that predicts kinetics of microbiome-meditated metabolism of a drug candidate.

With reference to FIG. 2C, at 230, the processor 110 may receive a chemical structure of a drug candidate as input. At 232, the processor 110 may predict as output whether the drug candidate will be metabolized by each of the plurality of microbiomes and the bacterial species in the non-transitory storage medium. The drug candidate may be represented in a chemical fingerprint. These chemical fingerprints may be used by a machine learning algorithm trained on the reference drugs to predict the metabolic steps performed by each microbiome and microbial species in the reference set.

### 2. Examples

The following discussion provides examples and experiments in connection with building and testing the system 100.

2.1 Materials

Bacterial strains were obtained from American Type Culture Collection, Deutsche Sammlung von Mikroorganismen and Zellkulturen, Biodefense and Emerging Infections Research Resources Repository and a lab strain collection. Human gut microbiomes were obtained from collaborators. Open-source software (QIIME) was used for microbiome analyses.

2.2 Reference Drug Selection

Beginning with the 1,760 FDA-approved drugs in the MicroSource Pharmakon library, a single UHPLC-qTOF MS method was optimized (C18 column; methanol:water gradient; positive ionization mode) that maximizes the number of detected drugs while minimizing run time. After eliminating antibiotics and non-oral drugs, drug molecular fingerprints were analyzed to identify 271 representative drugs that capture the greatest chemical diversity of the Pharmakon set as shown in FIG. 5A, where X axis is “PC1”, Y axis is “PC2”. These drugs, whose molecular weights vary from ˜155 to ˜800 Da, span a wide range of clinical indications. Many have limited or highly variable bioavailability. Other criteria (specific chemical groups, bioavailability, etc) may be used to select other reference drugs, or drugs may be selected from pre-clinical compound libraries.

2.3 Combinatorial Pooling

A computational simulation was performed to determine the number of inputs (drugs) that can be represented in increasing numbers of pools, as a function of changing parameters for replicates and overlap. One such pooling scheme assigns each of 271 drugs to a subset of 21 pools, such that each drug is placed in 4 pools but shares a pool with each other drug at most twice as shown in FIG. 5B. FIG. 5B illustrates combinatorial pooling of 271 drugs indicated in black circles into 21 pools (A-U). Pools V-X are no-drug controls. Other pooling schemes may alter the number of drugs, the number of pools, the number of pools each drug is placed in, or the overlap between the pooling patterns of each drug. Notably, the number of drugs that may be pooled scales approximately exponentially with the number of pools. In other words, increasing the number of pools less than 5-fold may scale the number of drugs from ˜250 to 25,000, using the same rules for replication and overlap.

Each of these pools was incubated with each of 60 human gut microbiome samples and 76 defined bacterial species/strains. Samples were collected over time. Liquid chromatography-coupled quadrupole time-of-flight mass spectrometry (MS) described above was used to quantify drug levels by targeted analysis. For each candidate drug-microbiome interaction, 4 pools provide quadruplicate replication for each drug in 4 distinct contexts of other drugs. The other pools serve as negative controls. In this manner, over 16,000 time-series profiles (271 drugs by 60 communities) were collected, each in quadruplicate, from only 21 drug pools.

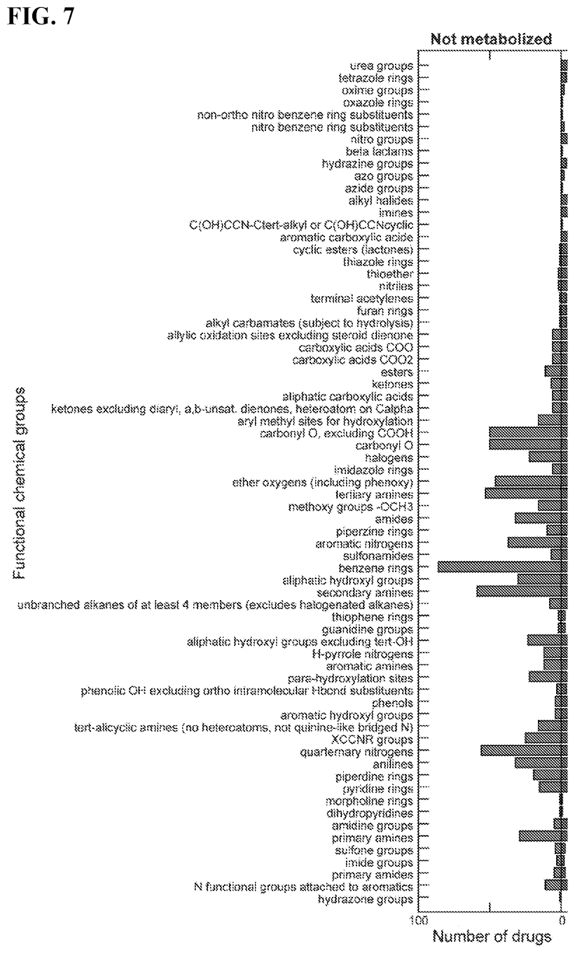

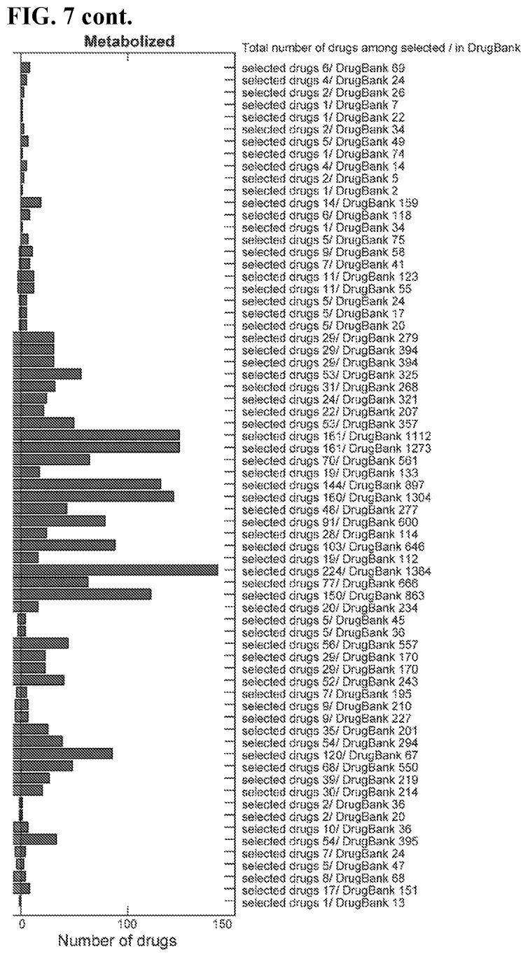

As a result, there were 176 drugs whose levels were significantly reduced by at least one individual's microbiota as shown in FIGS. 3 and 7A. Independent validation of single drugs confirms these results. Distribution of functional chemical groups in drugs that are metabolized or not metabolized across the 76 tested bacterial strains. Abundance of each chemical substructures among the 271 selected drugs and 2099 clinical drugs (from DrugBank) is indicated in FIG. 7 demonstrating that the functional group alone is not sufficient to predict microbiome drug metabolism and that the described computational predictions are required.

Furthermore, many drugs that do contain these substructures are protected from gut microbial activity as shown at the bottom of FIG. 7. These results establish that the determinants of microbiome-mediated drug modifications remain undefined.

Examples of drugs known to be metabolized by the human gut microbiome are sulfasalazine and risperidone. The time-series profiles of these two drugs from 11 individuals randomly selected from the 60 studied show the log₂ fold change in the amount of the drug being analyzed over a time span of 24 hours (FIG. 5C). Famcyclovir and tranilast are two drugs newly discovered to be metabolized by the human gut microbiome (FIG. 5D). The profile for tranilast exemplifies the interpersonal variability of the gut microbiome, as shown by the one individual that metabolized the drug to a much greater extent than the others.

2.4 Identification of Drug-Metabolizing Human Gut Commensal Species

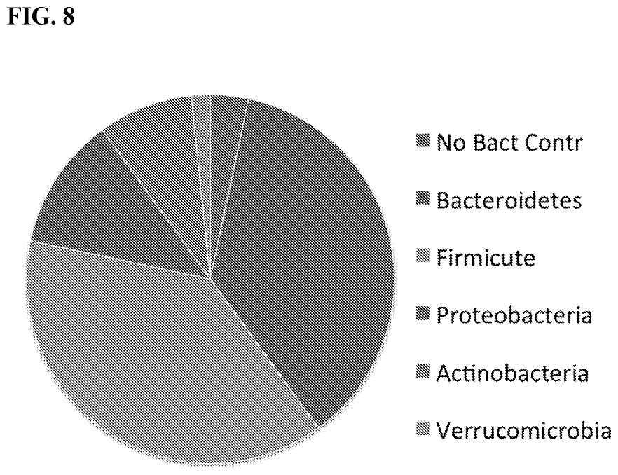

Using the combinatorial pooling method described above, each of the 21 drug pools was incubated, plus 3 no-drug control pools, with 76 defined human gut commensals from phyla shown in FIG. 8 grown in pure culture. Over 20,000 time-series profiles were collected, each in quadruplicate, of these specific drug-microbe interactions. In 97% of these profiles, drug levels were unchanged, suggesting that nonspecific reactions are unlikely.

2.5 Identification of Candidate Drug Metabolites

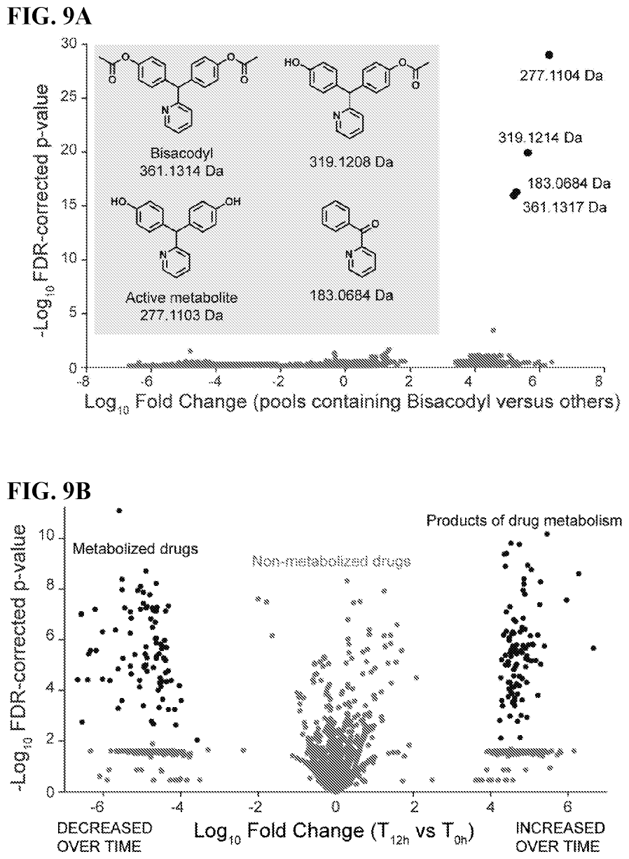

The reduced complexity of the pure-culture samples facilitates identification of drug metabolites by untargeted metabolomic analysis. These metabolites distinguish communities or species that metabolize the same drug in different ways and can provide insight into enzyme activities. LC-qTOF MS instrument detects several thousands of compounds (defined by distinct retention time and 2 detected m/z signals) in each sample. Metabolites of a drug occur only in pools that contain that specific drug. For example, across the 24 Clostridium asparaginoforme drug pools, only 4 metabolites are specifically present in the pools that contain bisacodyl (the prodrug form of a widely used laxative), as illustrated in FIG. 9A. With reference to FIG. 9A, C. asparaginoforme modifies bisacodyl. Each point represents a compound from the 24 drug pools (FIG. 5B) incubated with C. asparaginoforme. The x-axis represents the fold difference in abundance in bisacodyl-containing pools versus others at T₁₂ₕ and the y-axis provides the statistical significance. Compounds with significant enrichment in bisacodyl-containing pools are shown in black with observed mass noted, and others are in gray. The structures and predicted masses for bisacodyl and three metabolites are shown in the inset. Their observed masses are consistent with the expected masses of bisacodyl itself, two known ester hydrolysis products of bisacodyl (one of which is the active compound), and an unexpected metabolite with a size consistent with elimination of an entire phenyl acetate moiety from the drug, as illustrated in FIG. 9A. This 2-benzoylpyridine is not observed in other species that metabolize bisacodyl. The combinatorial pooling design uncovers specific metabolites of all drugs simultaneously as shown in FIG. 9B, and the dataset provides this information for all 74 tested species. FIG. 9B relates to all drug-specific metabolites produced by C. asparaginoforme. Each point represents a drug-specific metabolite (i.e., only metabolites observed across specific pools in a pattern that matches a single drug are shown). The x-axis provides average fold change between the T₁₂ₕ and T₀ₕ timepoints and the y-axis indicates the statistical significance. Compounds that change over time are shown black, others are in gray.

2.6 Identification of Microbiome-Encoded DMEs

Two independent strategies were developed to identify the microbiome-encoded DMEs responsible for these chemical transformations. For members of the Bacteroides (the most prominent genus in the human gut microbiome in the experiments), Proteobacteria, and other genetically tractable species, Insertion Sequencing (INSeq) is used to make mapped, arrayed transposon mutant libraries as described in Goodman 2009 and Goodman 2011. From these libraries, a representative set of mutants was identified that collectively include disruptions in most non-essential genes. Each mutant was incubated with the pool of drugs metabolized by the parent species. Genes required for drug metabolism by LC-MS were identified as above.

1,352 B. thetaiotaomicron mutants were selected from an arrayed library as described in Goodman 2009 that disrupt ˜70% of its non-essential genes. Each mutant was incubated with a pool of 30 drugs metabolized by this organism. Drug levels were measured over time as above. Genes necessary and sufficient for metabolism of 6 of the 30 drugs targeted by B. thetaiotaomicron were identified. FIGS. 10A-C show two examples: one of these is brivudine, an antiviral whose primary metabolite (bromovinyluracil; BVU) is dangerously toxic for patients taking 5-fluorouracil, a principal therapy for colorectal cancer. With reference to FIGS. 10A-C, transposon mutants representing disruptions in 70% of B. thetaiotaomicron (Bt) non-essential genes were screened for metabolism of 30 drugs targeted by the parent strain.

With reference to FIG. 10A, a mutant in the putative phosphorylase BT4554 (x) is unable to convert the antiviral drug brivudine into bromovinyluracil (BVU). Black points are mutants, sorted in the same order in both graphs; gray lines show background from no-bacteria control. FC, fold change. With reference to FIG. 10B, unmarked, nonpolar deletion of BT4554 recapitulates screen results.

With reference to FIG. 10C, a mutant in BT0152, a putative acetyl esterase, is unable to metabolize roxatidine acetate. Expression of BT0152 in E. coli (pbt0152) is sufficient to convert it into a roxatidine acetate metabolizer. FC refers to fold change. Ec refers to E. coli with an empty vector.

BVU from the related drug sorivudine killed 18 patients in this manner before being withdrawn from the market, but brivudine remains in use and the microbiome-encoded DME responsible has never been identified until now.

As a complementary approach, gain-of-function methods were developed to identify genes from human gut microbes that confer new drug metabolizing capabilities to a heterologous host such as E. coli. To this end, genomic DNA from any source species is fragmented to an average size of ˜3 kb, ligated into an expression vector, and transformed into E. coli. Colonies (˜40,000) are replicated onto duplicate agar trays in 384-grid format using a colony picker (QPix). The colonies from one copy of each tray are collected en masse by scraping and incubated with the pool of drugs metabolized by the source species. LC-MS analysis identifies trays that exhibit the ability to metabolize a drug and produce its metabolite(s). Colonies from the second copy of these trays are then pooled by rows and columns and analyzed as before to identify the specific E. coli clone that carries the functional DNA fragment from the source species. Using this approach, a B. thetaiotaomicron esterase was identified that targets the anti-hypertension drug diltiazem as illustrated in FIGS. 11A-G. FIG. 11A depicts the drugs metabolized by B. thetaiotaomicron and candidate drug metabolites identified by untargeted metabolomics. FIG. 11B shows the scheme for generation of an arrayed gain-of-function library and genome coverage in a representative B. thetaiotaomicron library. FIG. 11C illustrates the identification of active 384-well library plates that include clones with diltiazem deacetylation activity. FIG. 11D Shows the mapping of diltiazem-converting activity within active plates to identify active clones. FIG. 11E illustrates the mapping of active insert sequences to the B. thetaiotaomicron genome. FIG. 11A illustrates enzymatic validation using purified BT4096. FIG. 11G shows diltiazem-metabolizing activity of B. thetaiotaomicron wildtype, bt4096 mutant, and bt4096 complemented strains at three different expression levels (promoter strength: P2E5>P1E4>P2E3).

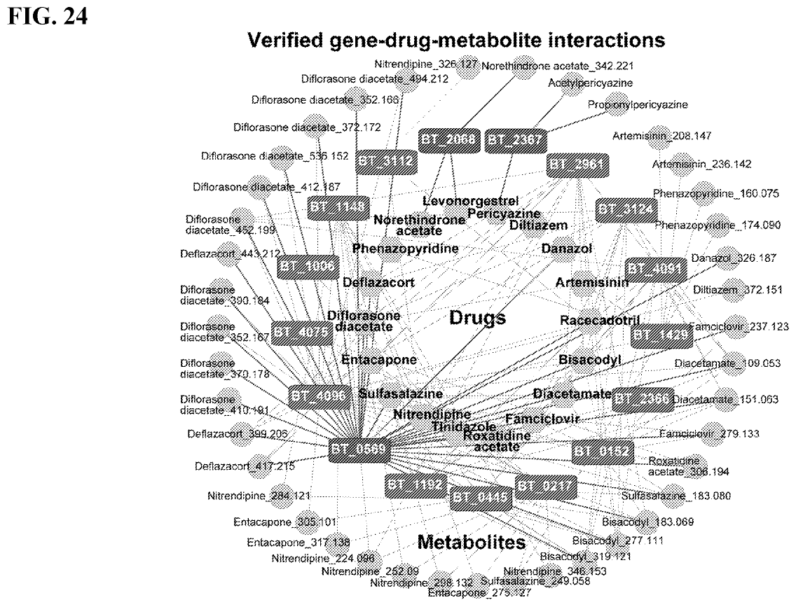

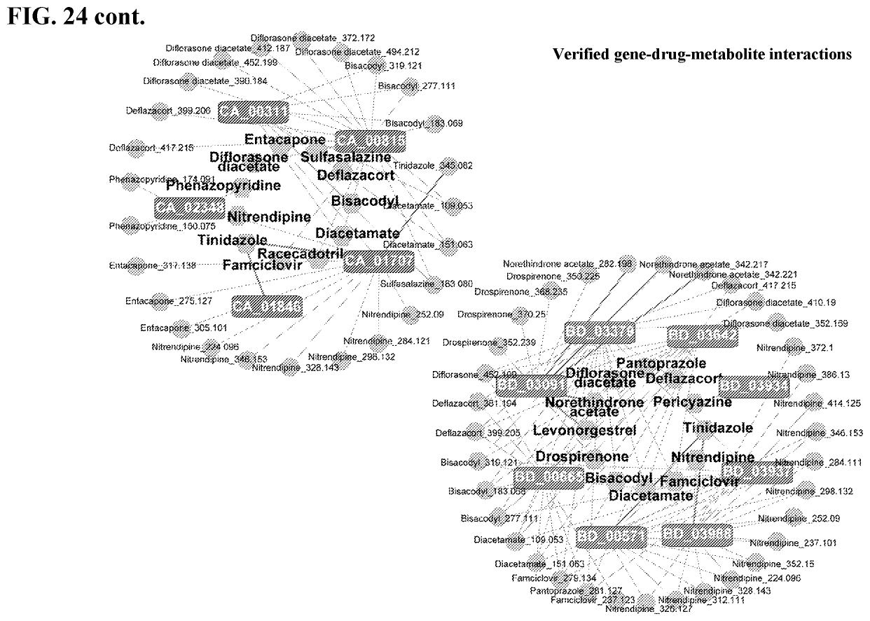

E. coli library generated B. thetaiotaomicron, B. dorei, and Collinsella aerofaciens captured genes 30 different genes that are collectively responsible for the metabolism of 22 different drugs as illustrated in FIG. 24. This strategy can identify redundant genes and does not require any genetic tools for the source species.

2.7 Prediction Of Microbiome Contribution to Drug and Metabolite Exposure Over Time

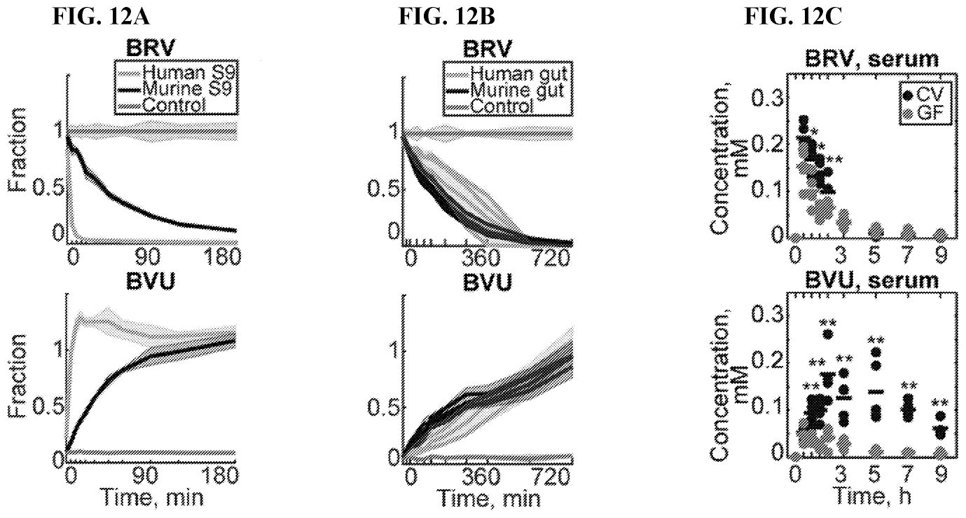

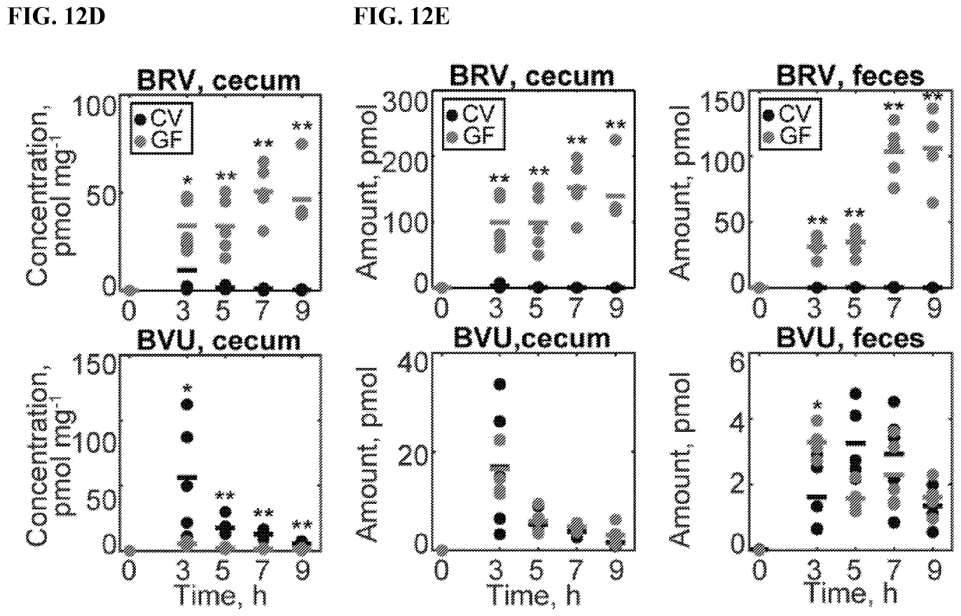

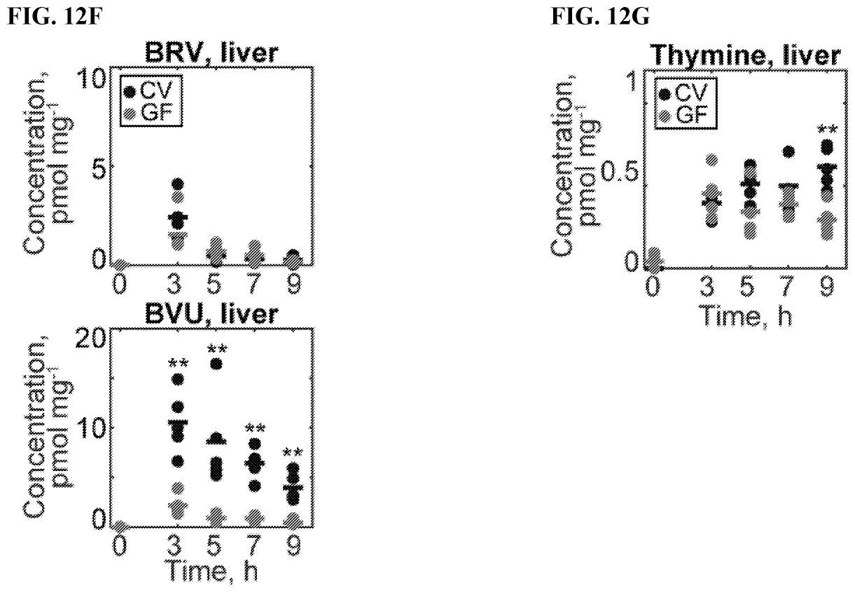

Brivudine (BRV) is an oral antiviral drug used in the treatment of shingles (herpes zoster) that is reported to be metabolized to bromovinyluracil (BVU) as shown in FIG. 4B by both host and microbiota. BVU is inactive against viruses but interferes with human pyrimidine metabolism, with lethal consequences for patients administered chemotherapeutic pyrimidine analogs such as 5-fluorouracil. Indeed, incubation of human and murine S9 liver fractions and unfractionated fecal microbial communities with BRV leads to stoichiometric conversion to BVU, confirming that both liver and microbiota are capable of this enzymatic transformation as illustrated in FIGS. 12A and B. FIG. 12A illustrates enzymatic conversion of BRV to BVU by human and murine S9 liver fractions. Shaded areas represent STD (n=5). FIG. 12B illustrates in vitro conversion of BRV to BVU by human and murine gut microbial communities. Lines and shading represent mean (n=4) and STD (n=16), respectively.

Oral BRV was administered to conventional (CV) and germfree (GF) C57BL/6 mice, and BRV and BVU concentrations were measured over time along the length of the gastrointestinal tract as shown in FIG. 4C. Serum kinetics of BRV and BVU in conventional (CV) and germ-free (GF) mice were compared following oral BRV administration.

To directly investigate microbial BVU generation in vivo, BRV and BVU concentrations are quantified along the intestinal tract over time as illustrated in FIG. 4C.

While CVR and GF mice exhibit similar BRV levels over time in the duodenum, drug concentrations are progressively reduced along the length of the CV gastrointestinal tract in agreement with prolonged exposure to increasing concentrations of gut bacteria.

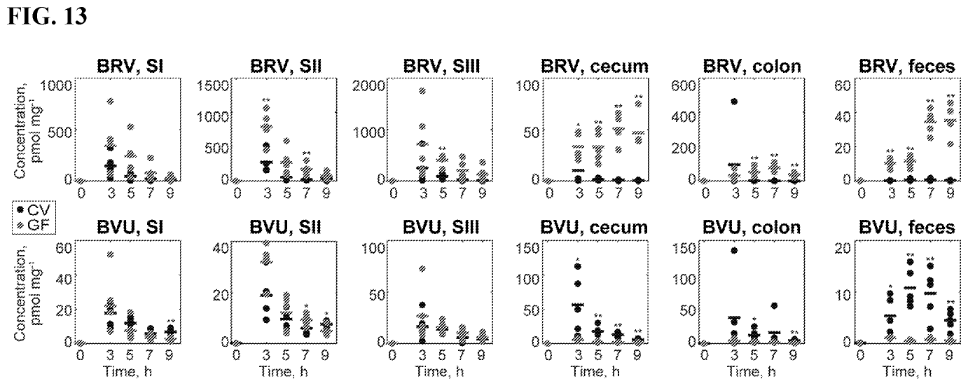

By contrast, GF mice maintain significantly higher BRV levels further along the gastrointestinal tract and in feces. BVU levels exhibit the opposite pattern, with increased intestinal concentrations in CV mice as compared to GF controls as illustrated in FIGS. 4C and 12D. Since GF animals have a larger cecum than their CV counterparts, the absolute amounts (rather than concentrations) of BRV and BVU in the large intestine are compared. The quantity of BVU in the feces of CV mice is insufficient to account for the amount of intestinal BRV metabolized. This discrepancy suggests that microbiome-produced BVU is absorbed from the lower intestine into systemic circulation as illustrated in FIG. 12E and FIG. 13. FIG. 13 illustrates BRV and BVU kinetics in intestinal compartments of CV and GF mice. For example, FIG. 13 illustrates concentration of BRV and BVU in intestinal compartments in GF and CV animals following oral BRV gavage. Horizontal lines show mean values of five animals. SI refers to duodenum, SII refers to jejunum, SIII refers to ileum, and *p≤0.05, **≤0.01 (t-test).

Indeed, CV animals exhibit significantly higher concentrations of BVU in serum than GF mice at later timepoints after drug administration as shown in FIG. 4D. CV mice accumulates higher levels of BVU in serum than their genetically identical GF counterparts, without a corresponding decrease in serum BRV, suggesting an intestinal (microbial) contribution to serum BVU as illustrated in FIG. 12C.

The increased concentration of serum BVU in CV as compared to GF mice is paralleled by increased BVU concentrations in the liver as illustrated in FIG. 12F.

BVU interferes with human pyrimidine metabolism by covalently binding to dihydropyrimidine dehydrogenase (DPD) in the liver, with lethal consequences for patients administered chemotherapeutic pyrimidine analogs such as 5-fluorouracil (5-FU). Notably, BRV-treated CV mice have higher BVU accumulation in the liver as shown in FIG. 4E and accumulate endogenous DPD substrates (e.g., thymine) in the liver, illustrating the contribution of the microbiota to toxicity under therapeutic regimes that do not involve 5-FU co-administration, and also illustrating the contribution of the microbiota to toxicity without 5-FU co-administration as illustrated in FIG. 12G. For all mouse data, horizontal lines show the mean of five animals and times reflect hours after oral BRV administration. SI refers to duodenum, SII refers to jejunum, SIII refers to ileum, and *p≤0.05, **≤0.01 (t-test).

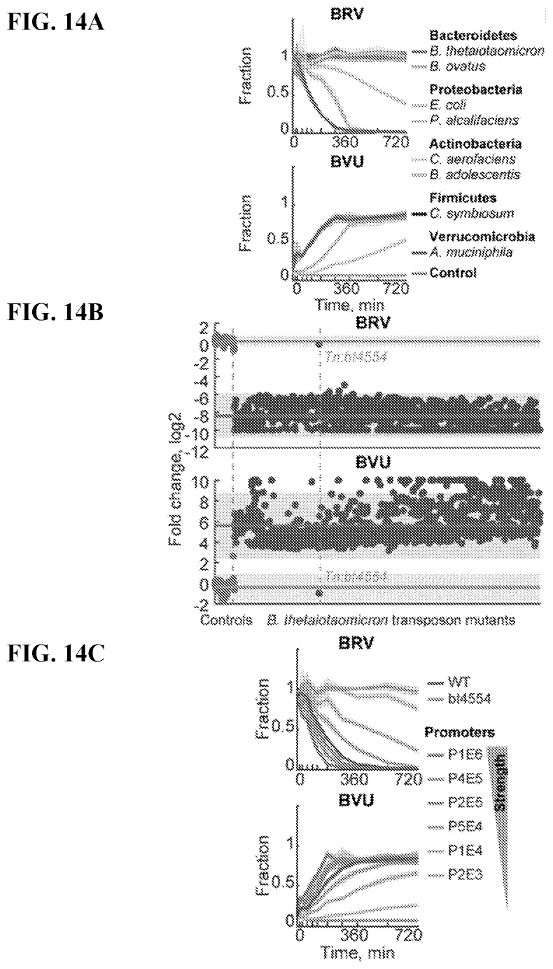

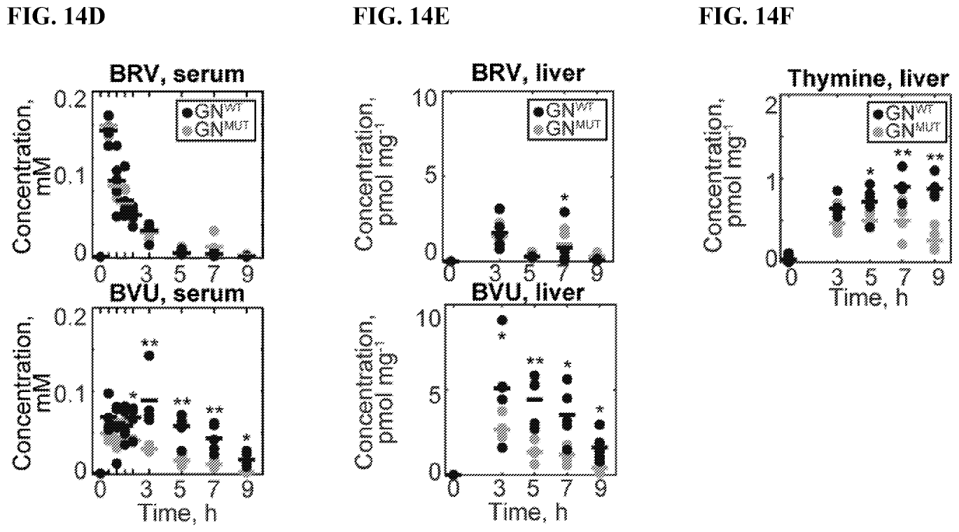

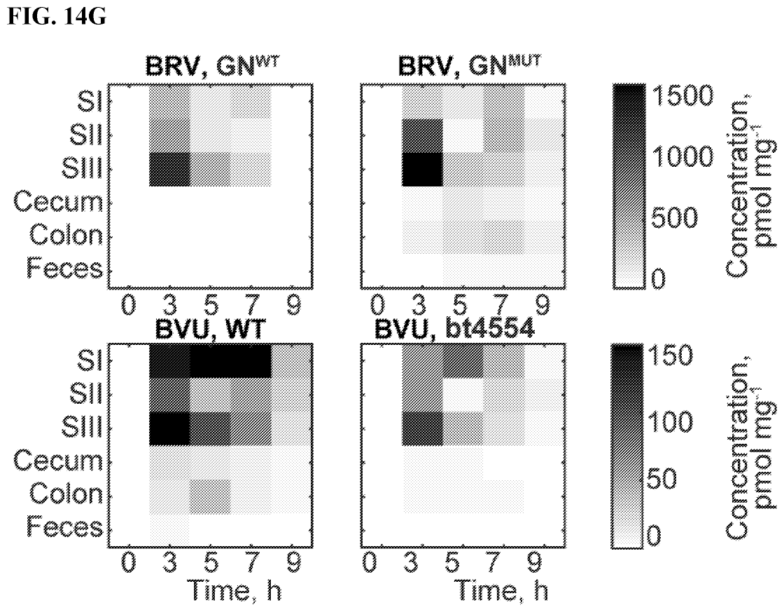

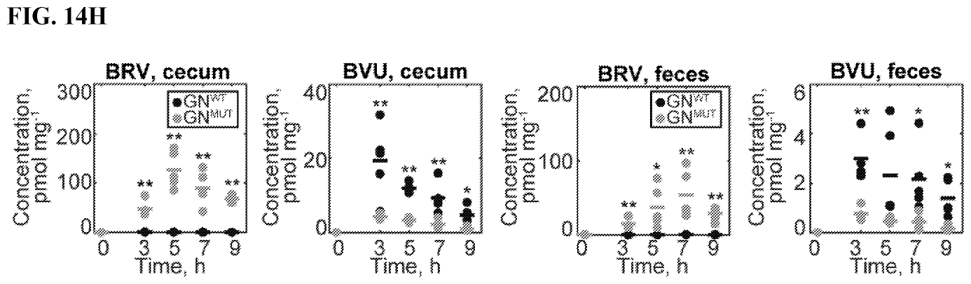

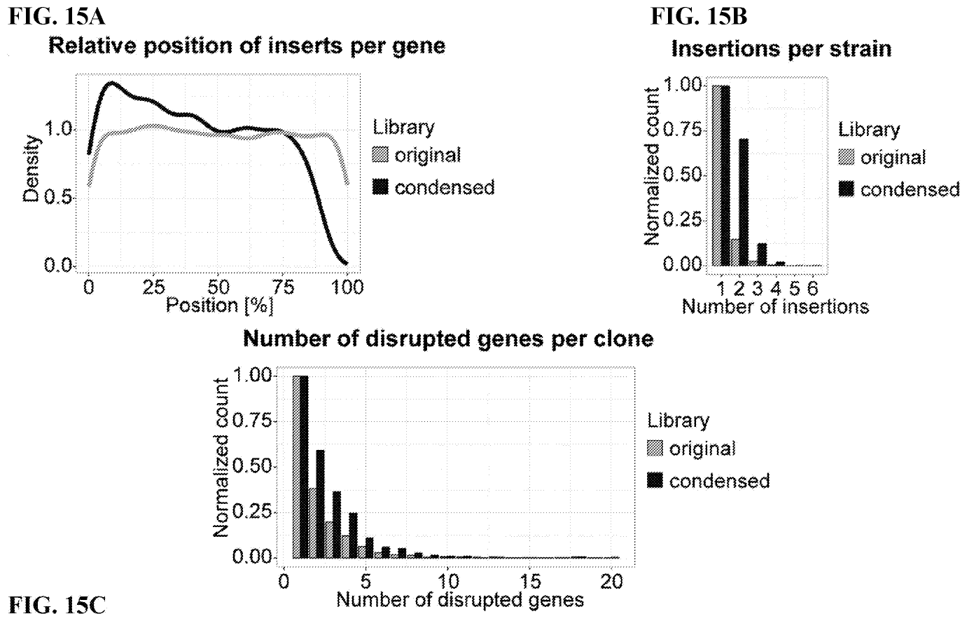

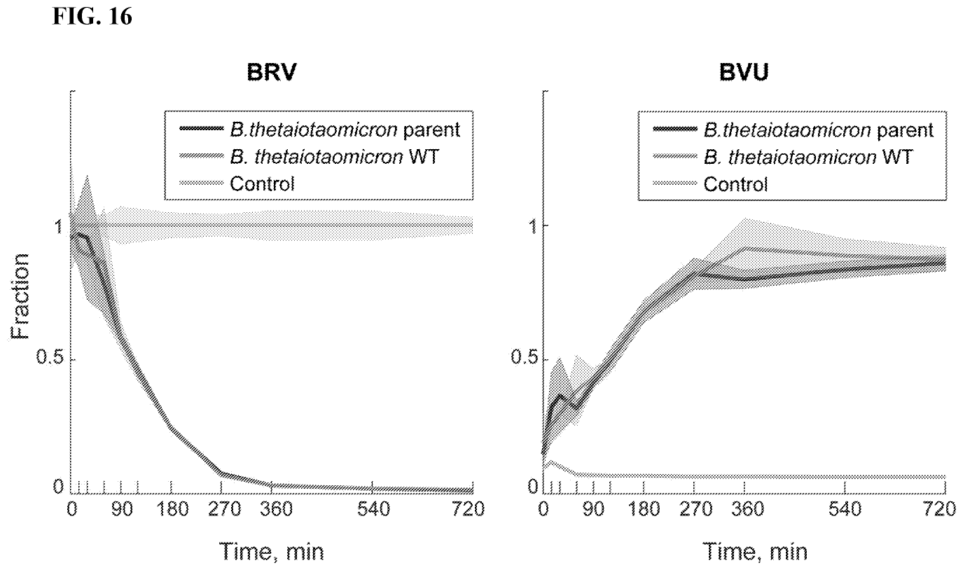

The contribution of microbial drug metabolism to serum drug and metabolite exposure was directly quantified by specifically modulating this activity in otherwise identical mice. To this end, the capacity of ten individual bacterial species was first determined, representing five major phyla that dominate the mammalian gut microbiota, for their capacity to convert BRV to BVU as illustrated in FIG. 14A. FIGS. 14A-H illustrate identification of a microbiome-encoded enzyme responsible for BRV metabolism and measurements of its contribution to pharmacokinetics and toxicity. Of these species, Bacteroides thetaiotaomicron and Bacteroides ovatus possess the highest metabolic activity, consistent with previous reports that members of this genus can metabolize the structurally similar drug sorivudine (SRV) as discussed in Koppel N, Maini Rekdal V, Balskus E P. Chemical transformation of xenobiotics by the human gut microbiota. Science. 2017; 356(6344). doi: 10.1126/science.aag2770. PubMed PMID: 28642381; PMCID: 5534341. These species also represent one of most abundant phyla and genera in the gut of humans and mice. To identify BRV-metabolizing enzymes in Bacteroides, a mapped, arrayed library of B. thetaiotaomicron VPI-5482 transposon mutants was condensed as discussed in Goodman 2009 to eliminate redundancy, resulting in 1290 strains that collectively disrupt expression of 2350 genes (˜75% of predicted non-essential genes) as illustrated in FIGS. 15A-C. Each of these strains was tested for the ability to metabolize BRV to BVU, and a single mutant is identified, carrying a transposon insertion in bt4554, that exhibits a loss of function phenotype as illustrated in FIGS. 10A-B and 14B. FIG. 14B illustrates log 2 fold change of BRV and BVU concentrations of B. thetaiotaomicron transposon insertion mutants (n=1290) compared to media controls (n=83) after 24 hours of incubation. Each point represents one strain, sorted along the x-axis in the same order in top (BRV) and bottom (BVU) panels. Mean fold changes and 95% prediction intervals for controls and strains are indicated by solid lines and shaded areas, respectively. Targeted gene deletion and complementation at different expression levels confirmed that bt4554, encoding a predicted purine nucleoside phosphorylase conserved among Bacteroides, is necessary and sufficient for BRV conversion to BVU and that its expression is the rate-limiting step in B. thetaiotaomicron BRV metabolism as illustrated in FIGS. 14C and 16. In FIG. 14A and FIG. 14C, lines and shaded areas depict the mean and STD of independent cultures (n=4-8). For all mouse data, horizontal lines show mean of five animals and times reflect hours after oral BRV administration, and *p≤0.05, **≤0.01 (t-test). FIG. 16 illustrates BRV to BVU conversion by B. thetaiotaomicron VPI-5482 parental and the tdk (WT) strain used as a genetic background for bt4554 deletion and complementation. Shaded areas depict the STD (n=4).

Germfree mice were colonized with the wildtype and mutant strains. BRV was administered. Drug and metabolite levels were monitored, over time, across different tissues and sera as shown in FIGS. 14D-H.

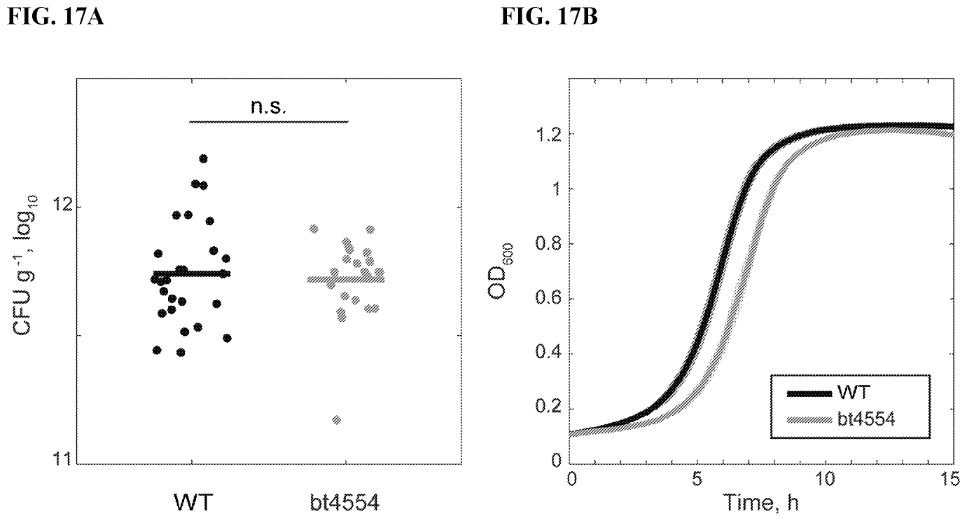

B. thetaiotaomicron wildtype and bt4554 mutant strains exhibit comparable growth rates in vitro and colonize GF mice at similar levels as illustrated in FIGS. 17A-B. FIGS. 17A-B relate tole vivo and in vitro growth of B. thetaiotaomicron wildtype and bt4554 strains. FIG. 17A illustrates fecal colony-forming unit (CFU) counts of GN^{WT} and GN^{MUT} mice 4 days after colonization (n>20). FIG. 17B illustrates kinetics of anaerobic growth in GMM. Shaded areas depict the STD (n=4).

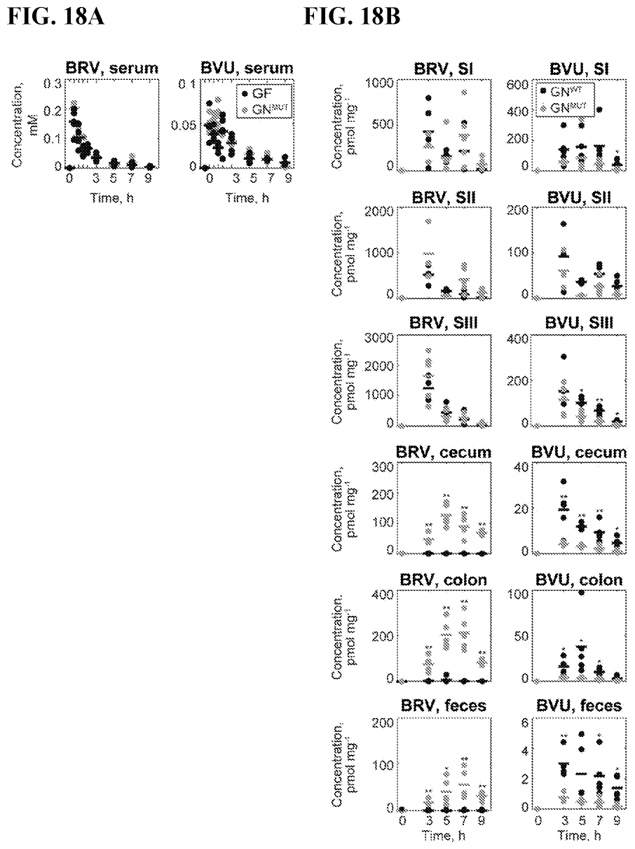

Administration of BRV to gnotobiotic (GN) mice mono-colonized with WT (GN^{WT}) or bt4554 mutant bacteria (GN^{MUT}) results in indistinguishable BRV serum kinetics, consistent with the physiological similarity between these animals and further suggesting that microbial BRV metabolizing activity in the intestine does not influence BRV bioavailability or systemic elimination. By contrast, serum BVU levels are significantly higher in GN^{WT} as compared to GN^{MUT} animals as illustrated in FIGS. 14D and 18A. FIG. 18A illustrates comparison of BRV and BVU serum kinetics between GF and GN^{MUT} mice. GN^{WT} mice also exhibit increased BVU levels and thymine accumulation in the liver after BRV administration as illustrated in FIGS. 14E-F. As observed in comparisons between CV and GF animals, increased systemic BVU exposure in GN^{WT} mice is paralleled by significant intestinal BRV metabolism as illustrated in FIGS. 14G and 18B. In FIG. 14G, each field represents the mean of five animals. FIG. 18B illustrates BRV and BVU kinetics in intestinal compartments of GN^{WT} and GN^{MUT} mice following oral BRV gavage. Horizontal lines show mean values of five animals of mixed gender. SI refers to duodenum, SII refers to jejunum, SIII refers to ileum, and *p≤0.05, **≤0.01 (t-test).

Because other aspects of host physiology, such as cecum size and intestinal transit time, are matched between GN^{WT} and GN^{MUT} animals, intestinal drug and metabolite concentrations can be directly compared and balanced. This reveals that wildtype B. thetaiotaomicron completely metabolizes cecal BRV, and the resulting BVU is almost entirely absorbed from both cecum and colon. By contrast, BRV is poorly absorbed from the lower intestine and GN^{MUT} mice excrete the drug in feces as illustrated in FIG. 14H.

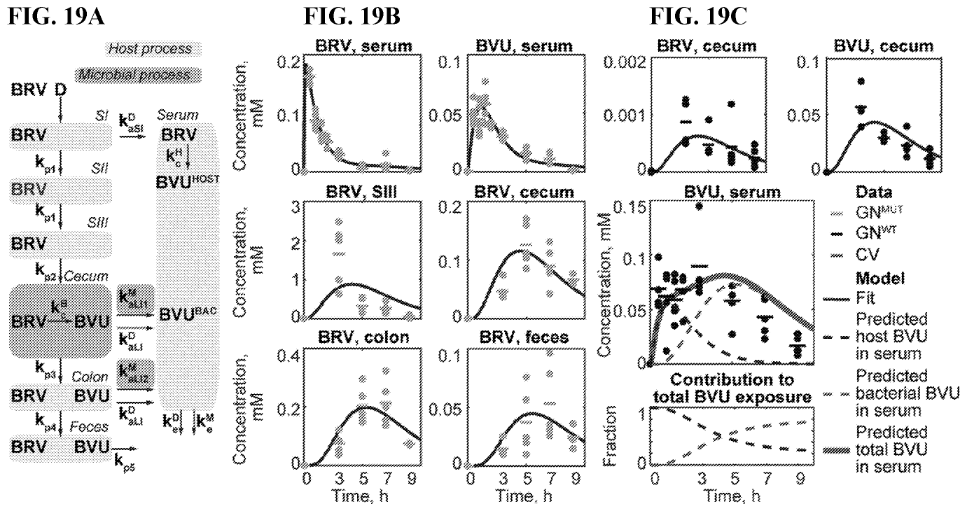

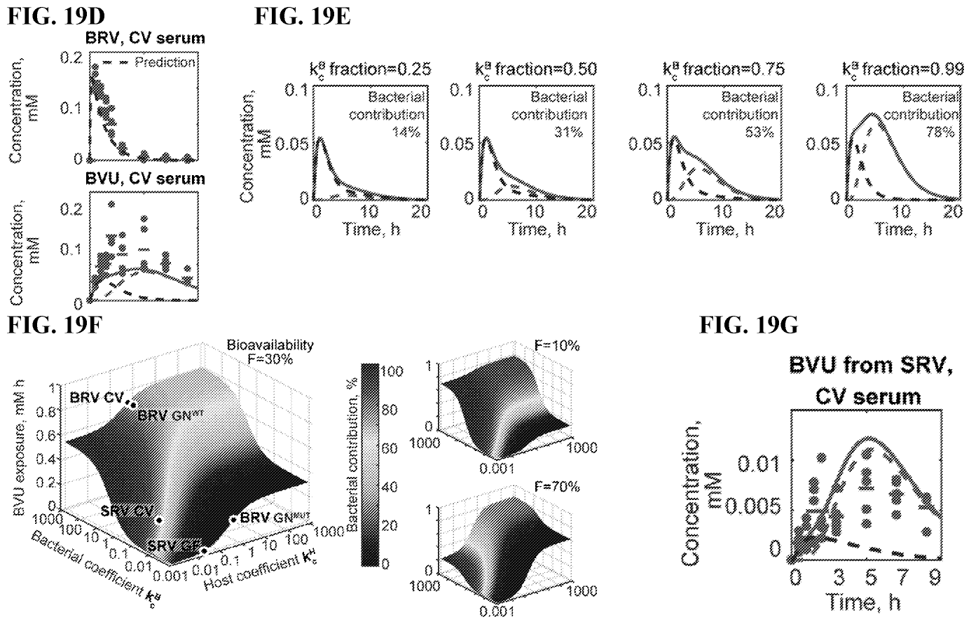

These quantitative measurements of drug and metabolite levels, collected over time, were used in various compartments, and in the presence and absence of microbial drug metabolism, to build the predictor module 130 which implements a physiologically based pharmacokinetic model to predict the levels and source of systemic drug and metabolite exposure over time, and quantify the contribution of host and microbiota to systemic drug and metabolite exposure. First, to parameterize processes independent from microbial BRV metabolism (grey compartments in FIG. 19A), a global optimization procedure was used to fit measured BRV and BVU kinetics in serum and intestinal compartments of GN^{MUT} mice. FIG. 19A illustrates schematic representation of compartments and sub-processes included in the physiologically based model of host and microbial contribution to BRV, SRV, and BVU pharmacokinetics. SI refers to duodenum, SII refers to jejunum, and SIII refers to ileum.

Measured BRV and BVU kinetics in serum and intestinal compartments of GN^{MUT} mice were used to parameterize processes independent from bacterial BRV conversion (grey compartments as illustrated in FIG. 19A) using the global optimization procedure to best fit the measured data. These processes include rates for i) BRV absorption from small and large intestine to blood (k_{aSI}^{D} and ka_{LI}^{D}); ii) BRV elimination (kₑ^{D}); iii) host BRV to BVU conversion (k_{c}^{H}); iv) BVU elimination (kₑ^{M}); and v) intestinal propagation/transit (kₚ₁ to kₚ₅) as illustrated in FIG. 19B.

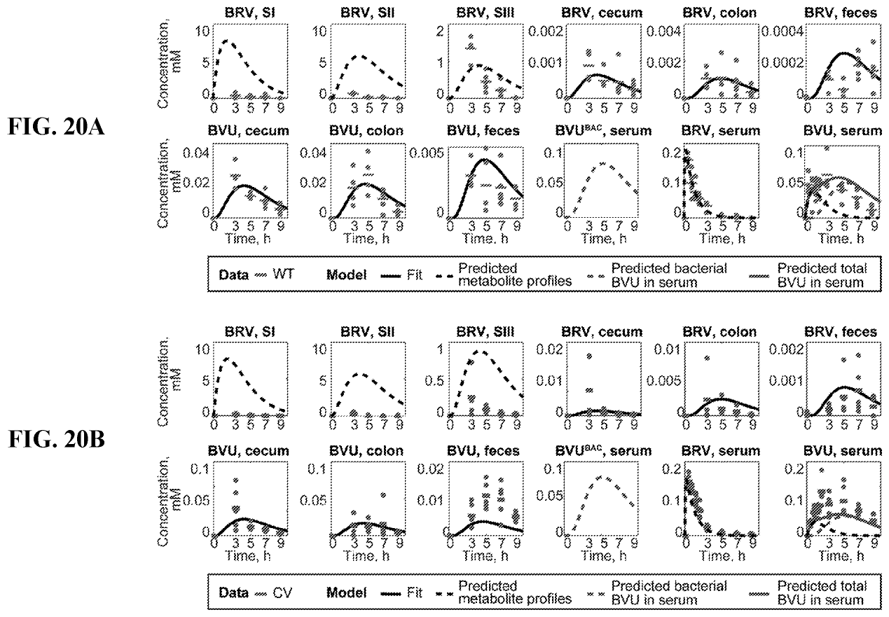

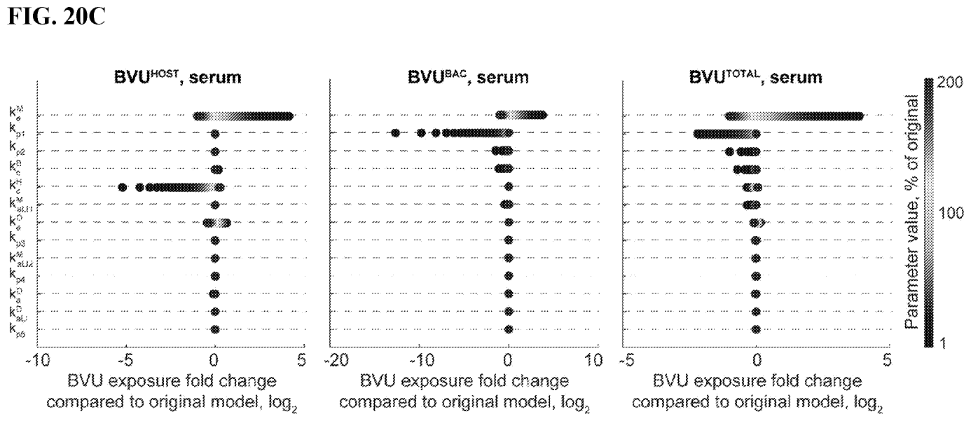

To parameterize processes dependent on microbial BRV metabolism (green compartments in FIG. 19A), including bacterial BRV to BVU conversion (k_{c}^{B}) and BVU absorption rates from cecum and colon (KaLI1^{M} and k_{aLI2}^{M}), measured BRV and BVU kinetics in cecum and colon (but not serum) of GN^{WT} mice, as illustrated in FIGS. 19C and 20A, are used. FIGS. 19C-E illustrate parameterization of microbiota-dependent intestinal drug metabolism and prediction of microbial and host contributions to serum BVU in GN^{WT} CV mice, predicting the impact of microbial drug metabolism rate on microbial contribution to serum BVU. FIGS. 20A-C illustrate parametrization of the pharmacokinetic model implemented by the prediction module 130 for BRV and BVU and sensitivity analysis. FIG. 20A illustrates fitting and prediction of BRV and BVU kinetics in different compartments of GN^{WT} mice following oral BRV administration. The nature of the small intestine data (SI-SIII) precludes using this data for fitting and comparison between the predicted and measured metabolic profiles, due to i) physiologically uneven distribution of alimental material and ii) sampling time points insufficient to monitor initial drug and metabolite dynamics.

The prediction module 130 accurately predicts BRV kinetics in serum of GN^{WT} mice (PCC=0.99). Further, the prediction module 130 predicts host and microbial contributions to serum BVU. The sum of these predicted contributions accurately matches total serum BVU measured in GN^{WT} animals (PCC=0.76) as illustrated in FIG. 19C. Comparison of the area under the curve for estimates of host and microbial contributions to serum BVU reveals that microbial activity accounts for nearly all of the serum BVU measured at later timepoints, and 77% of total BVU exposure in serum of GN^{WT} mice as illustrated in FIG. 19C.

This prediction module 130 that accurately predicts pharmacokinetics in GN^{WT} mice also applies to an unfractionated gut microbial community.

To predict the microbial contribution to serum BVU in context of a complex microbiota, model parameters of the predictor module 130 are altered to reflect BRV and BVU measurements collected from cecum and feces of CV mice as illustrated in FIGS. 19D and 20B. Despite increased microbial complexity, the predictor module 130 accurately predicts serum BRV kinetics in these animals. The sum of predicted host and microbial contributions to serum BVU matches the measured total serum BVU in CV animals (PCC=0.98 and 0.90, respectively) as illustrated in FIG. 19D.

Accordingly, host and microbial contributions to serum drug and metabolite levels are predicted even in cases where the responsible microbiome-encoded enzyme is unknown.

Sensitivity analysis, which estimates the impact of varying each of the 13 rates included in the model on serum BVU exposure, reveals that the parameters that most effect host and microbial contributions to serum BVU are distinct and that overall serum exposure is dependent on both host and microbial drug metabolic activity as illustrated in FIG. 20C. FIG. 20C illustrates normalized sensitivity analysis of the fully parameterized pharmacokinetic model for total BVU serum levels, and relative BVU serum contributions by the host and microbes. Model parameters fit for GN^{WT} were used as base values and the sensitivity of host-derived, microbe-derived, and total BVU to each parameter was assessed by calculating the relative change in serum BVU exposure after stepwise changes of each parameter separately in the range of 1% to 200% of its base value. Kinetic rates are as follows: ke^{M} refers to systemic elimination of the drug metabolite; kₚ₁ to kₚ₅ refer to intestinal transit/propagation; kc^{B} refers to drug conversion by intestinal microbes; k_{c}^{H} refers to drug conversion by the host; k_{aLI1}^{M} and k_{aLI2}^{M} refer to metabolite absorption from large intestine; k_{c}^{D} refers to systemic elimination of the drug; kₐ^{D} refers to drug absorption from small intestine; and k_{aLI}^{D} refers to drug absorption from large intestine.

Interpersonal differences in microbial community composition likely alters the BRV metabolism capacity of these communities as illustrated in FIG. 5D. The fully parameterized pharmacokinetic model was used to simulate how differences in microbial BRV metabolism rates, expressed as a fraction of total BRV metabolizing activity (k_{c}^{B}/(k_{c}^{B}+k_{c}^{H})) impact the microbiome contribution to serum BVU kinetics and cumulative exposure when host BRV metabolism is kept constant. For example, simulating interpersonal differences in gut community composition or antibiotic exposure by changing bacterial drug conversion rate (k_{c}^{B}) reveals that the predicted microbiome contribution to serum BVU can vary from 0% to 78%, and that total systemic BVU exposure can vary more than four-fold, in response to this parameter as illustrated in FIG. 19E. Multiple parameters can also be altered simultaneously to predict the pharmacokinetics of other drugs that are subject to different bioavailability, host-and microbiome-mediated drug metabolism, and drug/metabolite absorption.

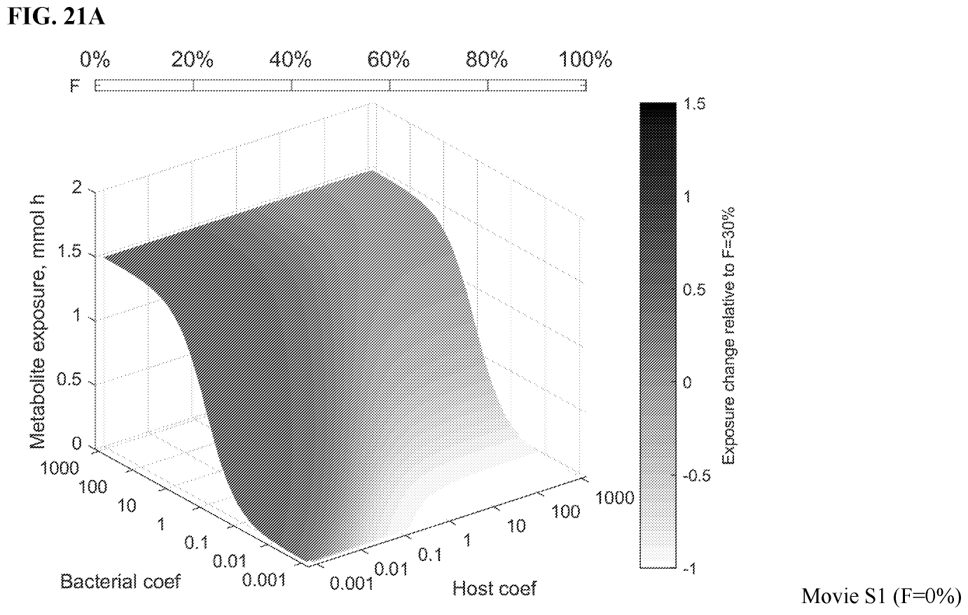

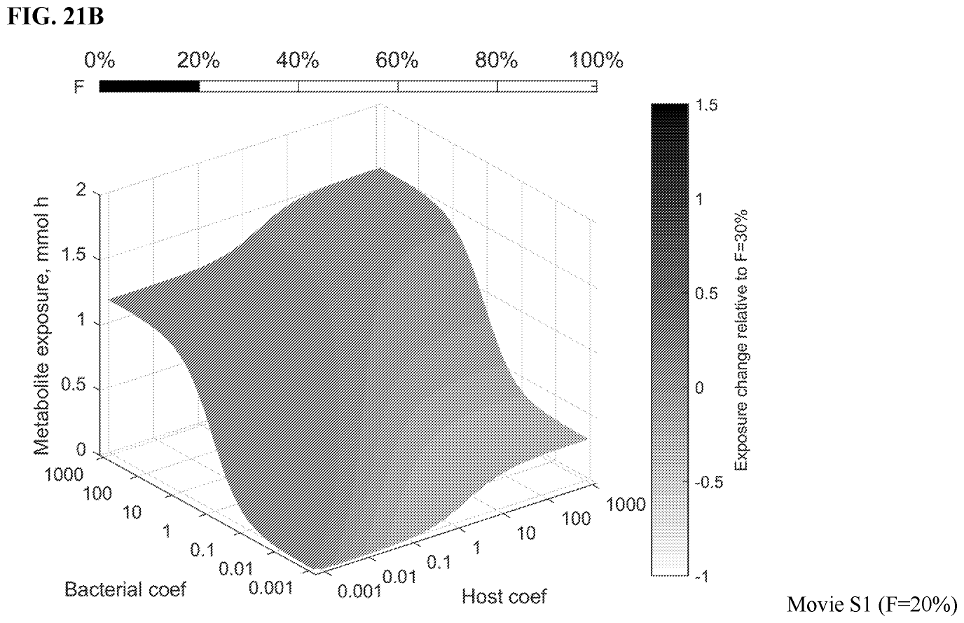

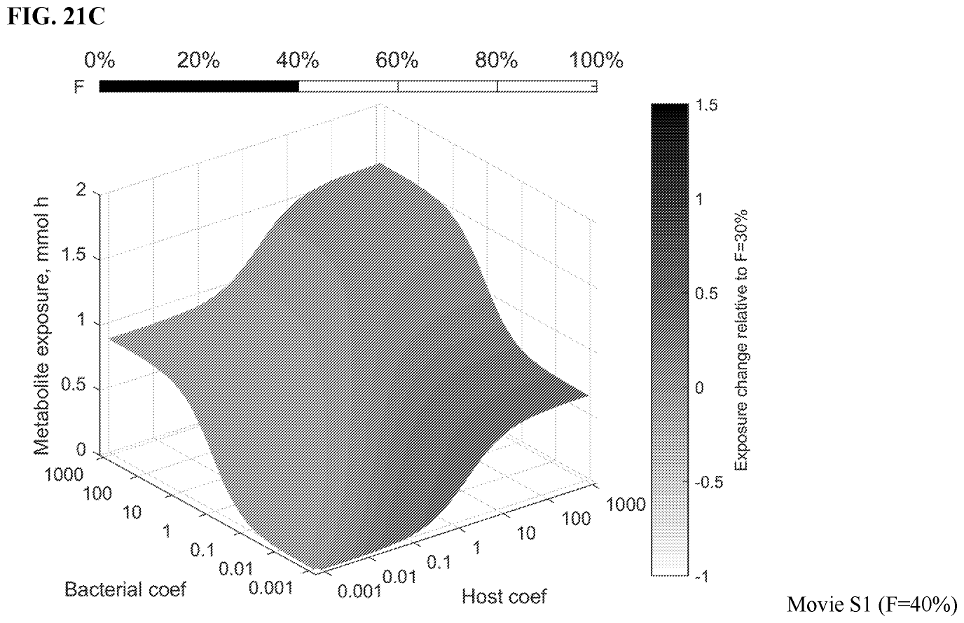

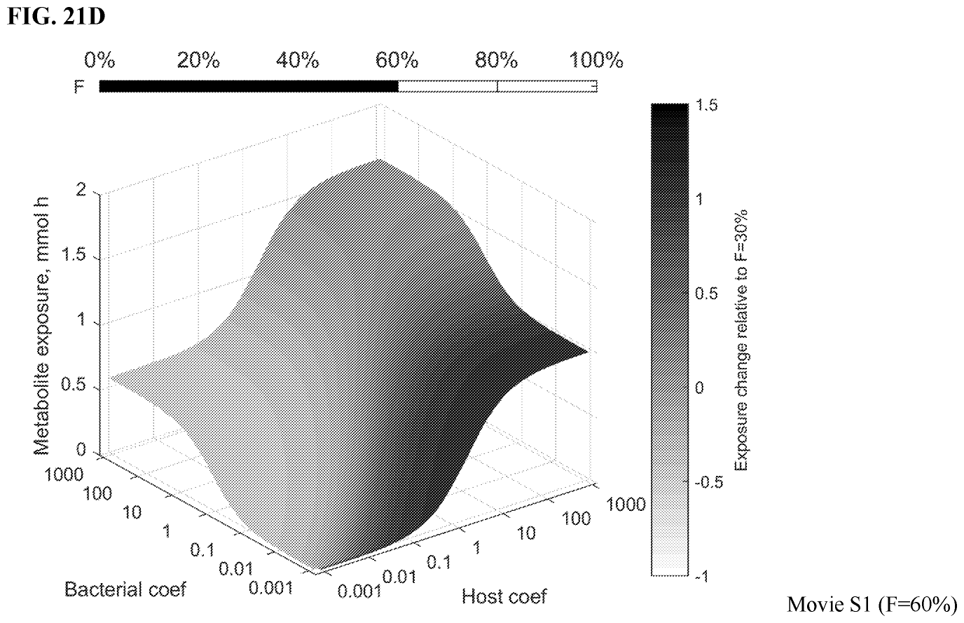

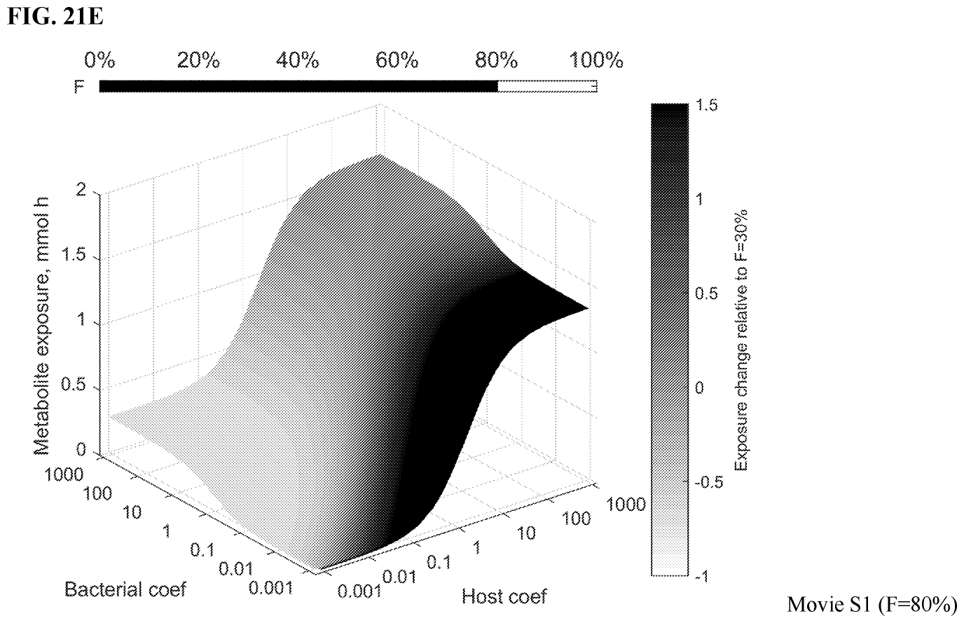

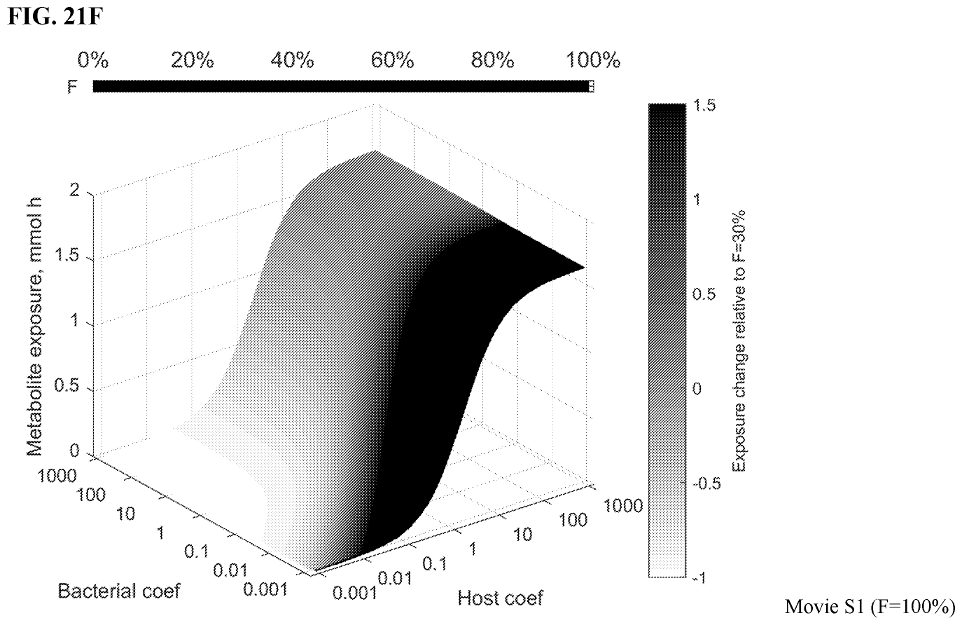

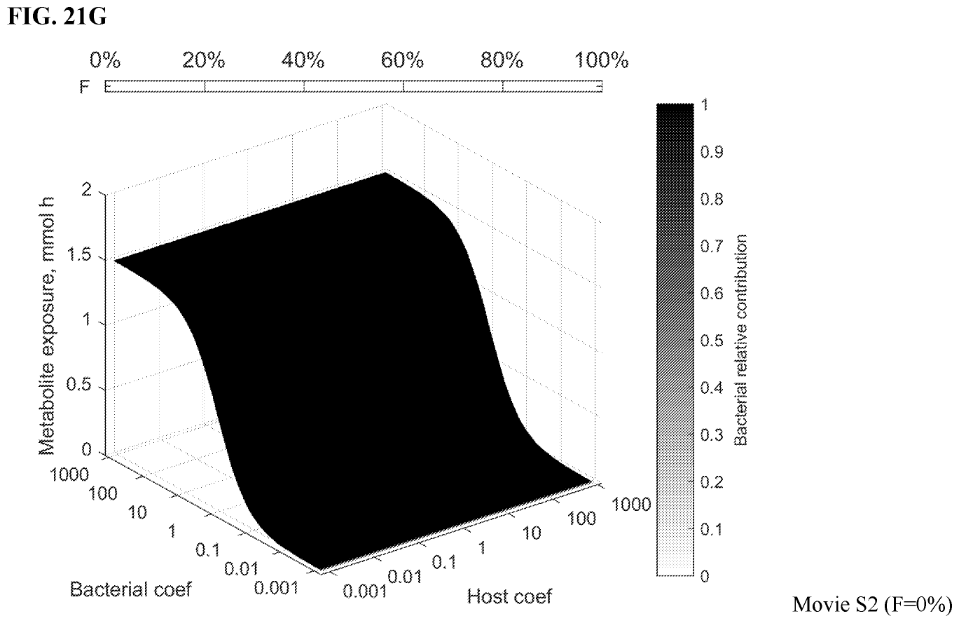

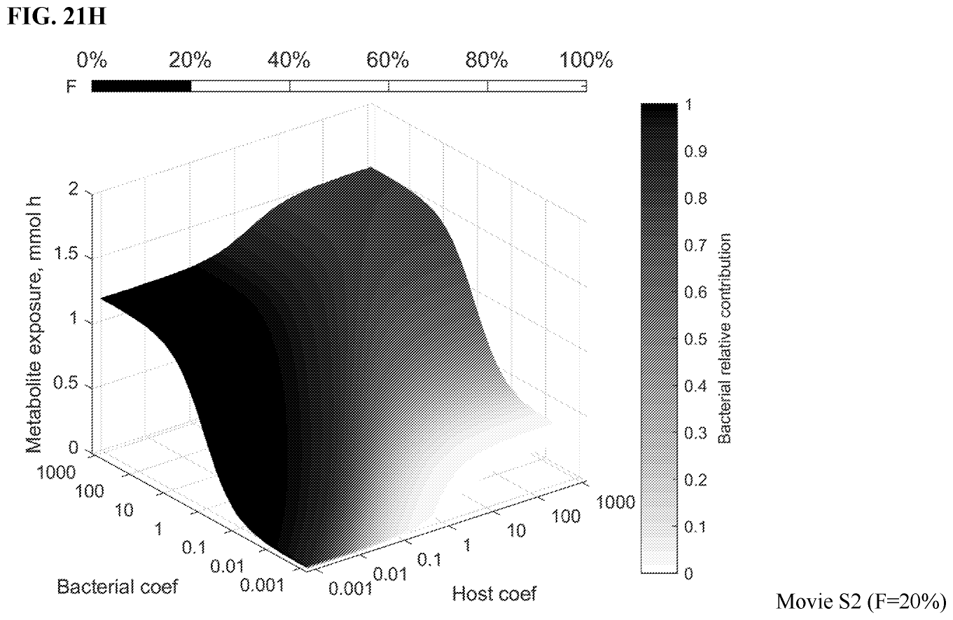

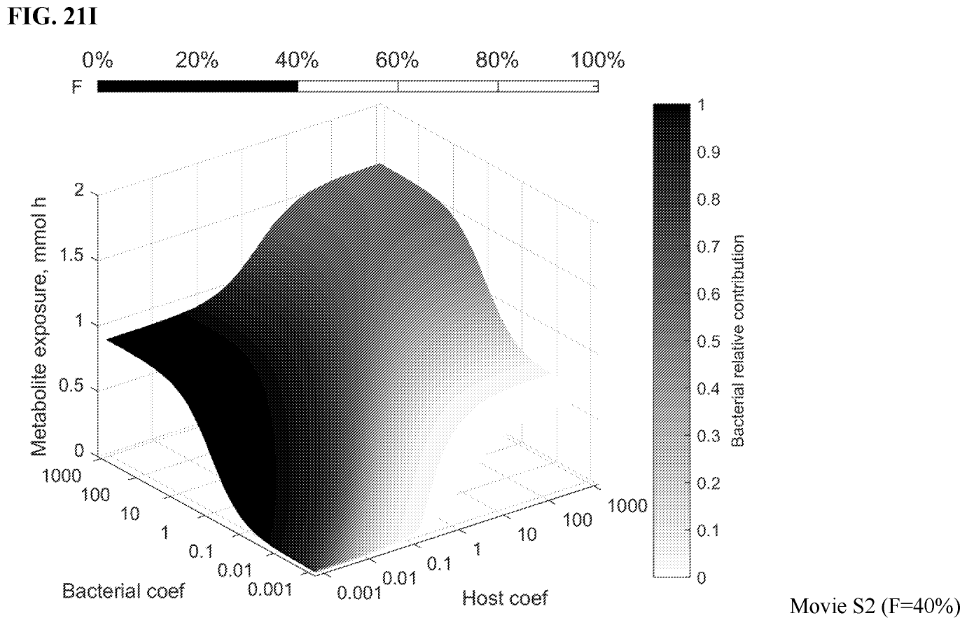

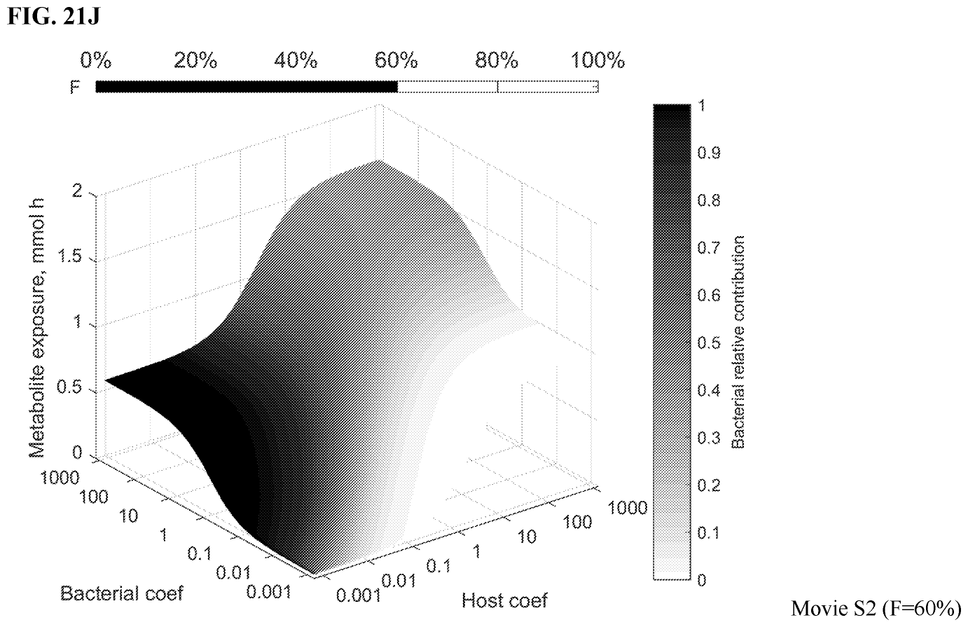

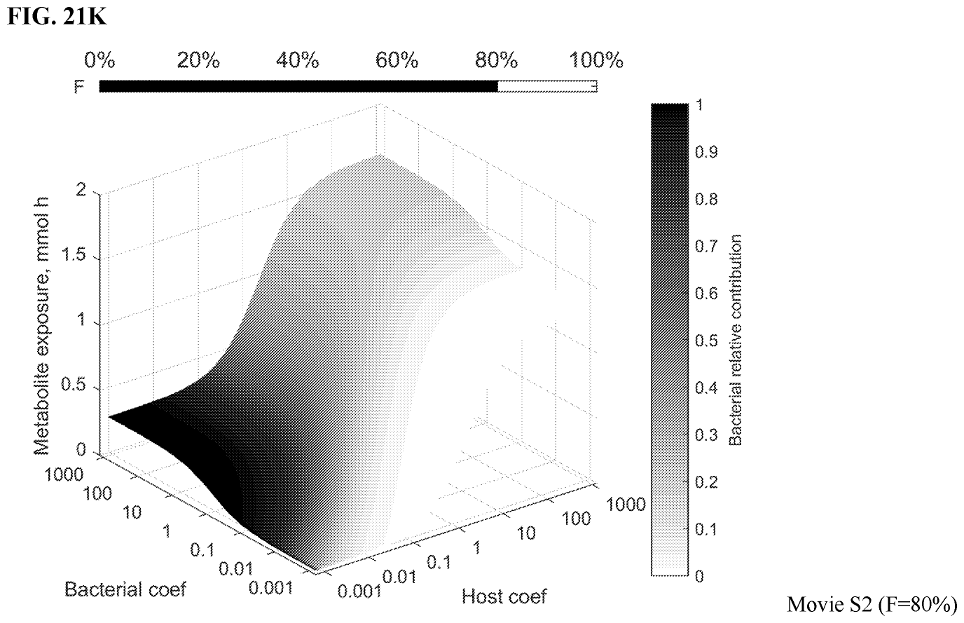

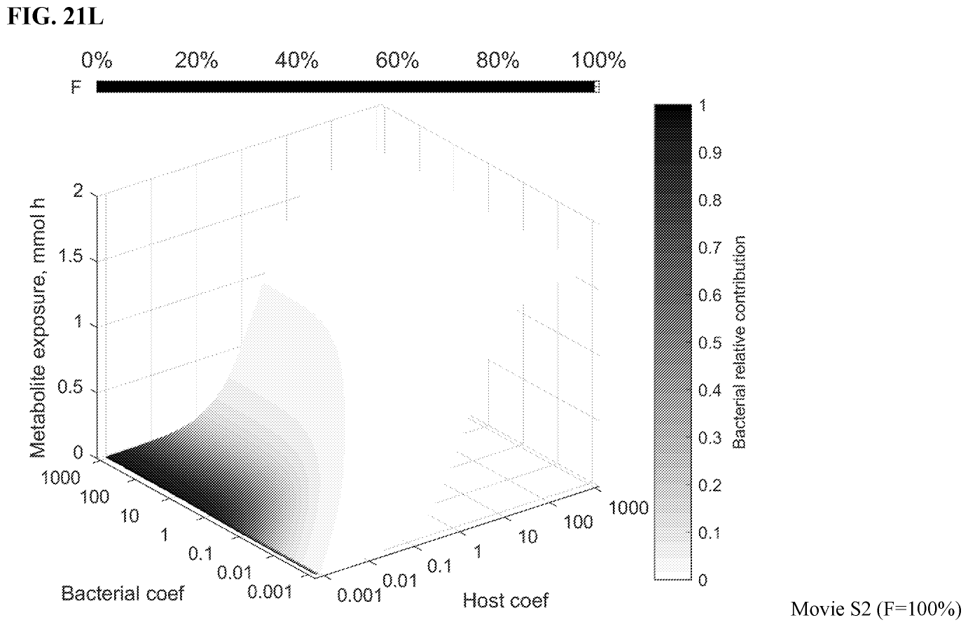

Next, the response of the model to simultaneous variation of both microbiome and host drug metabolizing activity was examined. For example, simultaneous alteration of parameters for both host and microbiome-mediated drug metabolism produces a 3-dimensional surface that estimates total serum metabolite exposure and relative microbiome contribution as a function of both parameters as illustrated in FIG. 19F. The predictor module 130 further reveals how bioavailability impacts these estimates at various host and microbiome drug metabolism rates as illustrated in FIGS. 19F and 21A-L. FIGS. 21A-F illustrate simulation of absolute metabolite exposure and relative microbial contribution to serum metabolite exposure as a function of host and microbiome metabolic capacity (k_{c}^{H} and k_{c}^{B}, respectively) and different drug bioavailabilities. FIGS. 21G-L illustrate simulation of absolute metabolite exposure as a function of host and microbiome metabolic capacity (k_{c}^{H} and k_{c}^{B}, respectively) and different drug bioavailabilities. For each combination of k_{c}^{H} and k_{c}^{B}, surface coloring depicts the relative change of serum metabolite exposure for a given drug bioavailability compared to 30% drug bioavailability.

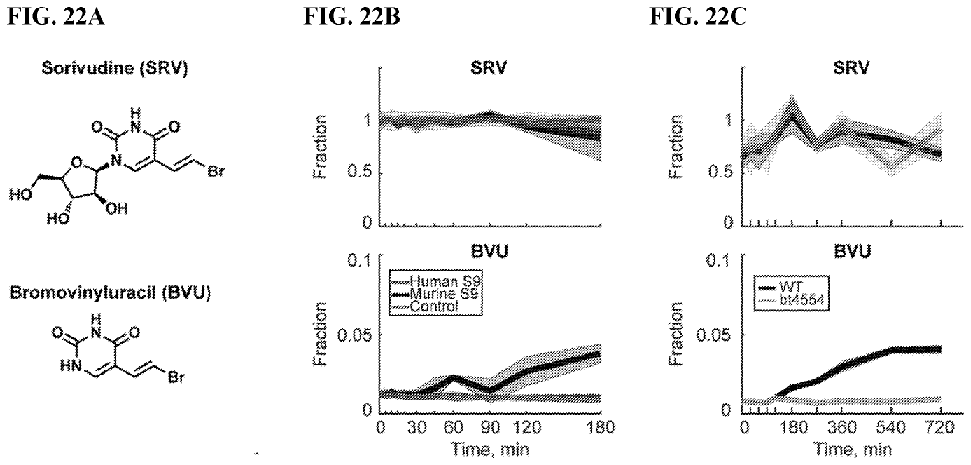

To test the predictions by the predictor module 130, sorivudine (SRV) is focused on, which is structurally similar to BRV but is metabolized to BVU at different rates by both the host and the microbiome, as illustrated in FIGS. 22A-C. FIGS. 22A-C illustrate in vitro characterization of microbial and mammalian sorivudine (SRV) metabolism. FIG. 22A illustrates chemical structure of SRV and BVU. FIG. 22B illustrates that enzymatic assays demonstrate that SRV is slowly converted to BVU by human and murine S9 liver fractions. Shaded areas represent STD (n=5). FIG. 22C illustrates that B. thetaiotaomicron slowly converts SRV to BVU in a BT4554-dependent manner.

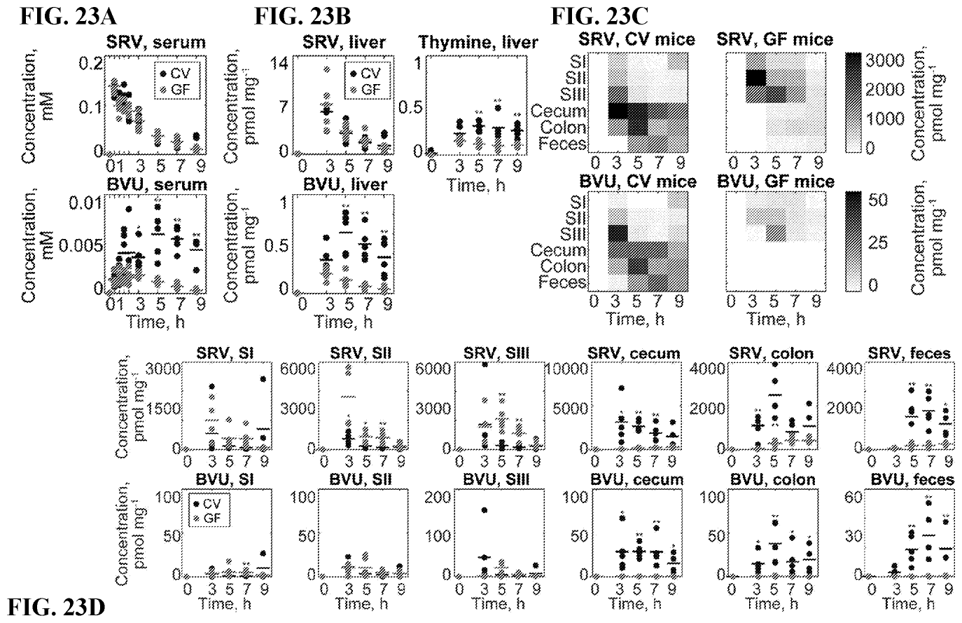

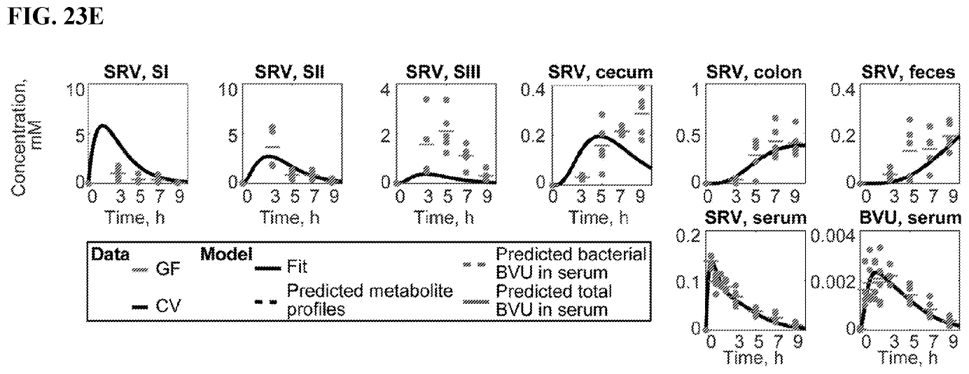

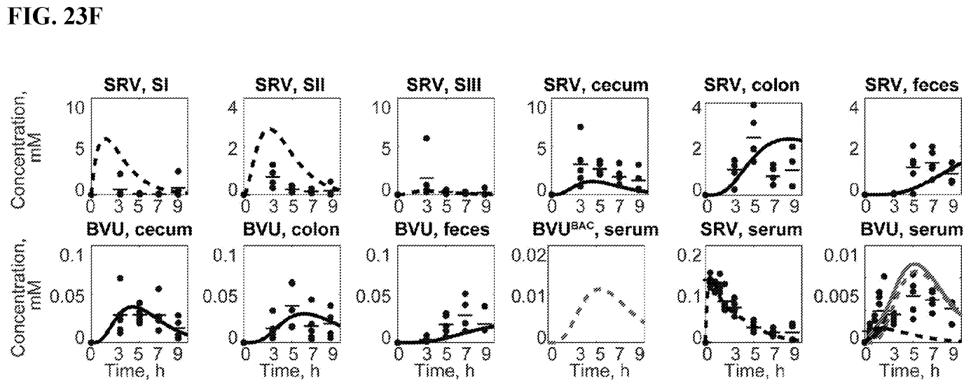

SRV is orally administered to CV and GF mice. Drug and metabolite levels are measured across tissues and over time as above. Serum drug and intestinal drug and metabolite measurements are provided as inputs to the predictor module 130. Notably, predicted serum metabolite kinetics match experimental measurements of serum BVU levels in SRV-treated mice (PCC=0.89), and also reveal the relative contribution of host and microbial SRV metabolizing activity to this exposure as illustrated in FIGS. 19F-G, 23A and 23B. FIG. 19G illustrates predicting host and microbial contribution to serum BVU after oral SRV administration to CV mice. Horizontal lines show mean of five animals and times reflect hours after oral SRV administration. FIGS. 23A-F illustrate in vivo characterization of SRV metabolism in CV and GF mice and quantification of microbial contribution to BVU serum exposure after SRV administration using pharmacokinetic modeling.

These results demonstrate that the predictor module 130 predicts both levels and sources of metabolite exposure for a drug subject to different host and microbiome drug metabolizing activity than BRV. FIG. 23C illustrates intestinal SRV and BVU concentrations over time. Each field represents the mean value of five animals. FIG. 23D illustrates SRV and BVU kinetics in intestinal compartments of CV and GF mice. FIG. 23E illustrates parameterization of microbiota-independent SRV kinetics using measurements from GF mice. FIG. 23F illustrates predicting microbial and host contributions to serum BVU in CV mice after SRV administration. BVU^{BAC} is the predicted bacterial contribution to serum BVU. For all mouse data, horizontal lines show the mean of five animals and times reflect hours after oral SRV administration. SI refers to duodenum, SII refers to jejunum, SIII refers to ileum, and *p≤0.05, **≤0.01 (t-test).

The model implemented by the predictor module 130 may be further elaborated to predict how other variables, such as bioavailability, impact the host vs. microbial contribution to serum drug or metabolite exposure as illustrated in FIG. 19F.

2.7.1 Chemicals

Brivudine, sorivudine, and 5,6-dihydrouracil were purchased from Santa Cruz Biotechnology, LC-MS grade solvents from Fisher Scientific, and all other chemicals from Sigma Aldrich, if not specified otherwise.

2.7.2 Bacterial Culture Conditions

Escherichia coli S-17λ pir strains are grown at 37° C. in LB medium supplemented with carbenicillin 50 μg/mL. B. thetaiotaomicron VPI-5482 (ATCC 29148) derived strains are grown anaerobically at 37° C. in liquid TYG medium. All anaerobic culturing is performed on brain-heart-infusion (BHI; Becton Dickinson) agar supplemented with 10% horse blood (Quad Five Co.). Cultures of bacterial gut communities and isolates for drug degradation assays are grown in Gut Microbiota Medium (GMM). For selection, gentamicin 200 μg/mL, erythromycin 25 μg/mL, and/or 5-fluoro-2-deoxy-uridine (FUdR) 200 μg/mL are added as indicated. A flexible anaerobic chamber (Coy Laboratory Products) containing 20% CO₂, 10% H₂, and 70% N₂ is used for all anaerobic microbiology steps.

B. thetaiotaomicron wild type and bt4554 are grown aerobically in 200 μL GMM (Table 1) in flat-bottom 96-well plates (Corning Incorporated) inoculated with 2 μL of overnight cultures in the same medium. Growth is monitored by OD600 measurements every 10 min (Eon microplate photospectrometer, Biotek).

TABLE 1

| | | |
|---|---|---|
| Component | Amount/L | Concentration |

| Tryptone Peptone | 2 | g | 0.2% | |
|---|---|---|---|---|
| Yeast Extract | 1 | g | 0.1% | |
| D-glucose | 0.4 | g | 2.2 | mM |
| L-cysteine | 0.5 | g | 3.2 | mM |
| Cellobiose | 1 | g | 2.9 | mM |
| Maltose | 1 | g | 2.8 | mM |
| Fructose | 1 | g | 2.2 | mM |
| Meat Extract | 5 | g | 0.5% | |
| KH₂PO₄ | 100 | mL | 100 | mM |
| MgSO₄—7H₂O | 0.002 | g | 0.008 | mM |
| NaHCO₃ | 0.4 | g | 4.8 | mM |
| NaCl₂ | 0.08 | g | 1.37 | mM |
| CaCl2 | 1 | mL | 0.80% | |
| Vitamin K (menadione) | 1 | mL | 5.8 | mM |
| FeSO₄ | 1 | mL | 1.44 | mM |
| Histidine Hematin Solution | 1 | mL | 0.1% | |
| Tween 80 | 2 | mL | 0.05% | |
| ATCC Vitamin Mix | 10 | mL | 1% | |
| ATCC Trace Mineral Mix | 10 | mL | 1% | |
| Acetic acid | 1.7 | mL | 30 | mM |
| Isovaleric acid | 0.1 | mL | 1 | mM |
| Propionic acid | 2 | mL | 8 | mM |
| Butyric acid | 2 | mL | 4 | mM |
| Resazurin | 4 | mL | 4 | mM |

| | | | |
|---|---|---|---|
| Noble Agar | 12 | g | 1.2% |

2.7.3 Construction of B. thetaiotaomicron Targeted Mutants

B. thetaiotaomicron tdk is indistinguishable from its parent strain with respect to BRV to BVU conversion as illustrated in FIG. 16. A counter-selectable allelic exchange procedure is utilized to generate in-frame, unmarked deletions in a B. thetaiotaomicron VPI-5482 tdk background (wild type; WT). In brief, ˜1,000 basepair (bp) regions flanking bt4554 are amplified with high-fidelity polymerase (HiFi HotStart ReadyMix, KAPA Biosystems), purified (Qiaquick PCR purification kit, Qiagen), and joined via Splicing by Overlap Extension (SOE) PCR (primers 1 and 4, Table 2). The product is gel purified (Qiaquick gel purification kit, Qiagen), cloned into the pExchange-tdk suicide plasmid using BamHI and XbaI restriction sites (New England BioLabs), and electroporated into E. coli S17λ pir. Sequence-verified constructs (primers 5 and 6, Table 2) are introduced into B. thetaiotaomicron tdk via conjugation. Merodiploid cells are selected for erythromycin resistance, colony purified and counter-selected for resistance to FUdR (Sigma Aldrich). Resolved clones are screened by PCR (Taq HotMaster Mix, QuantaBio) using primers 7 and 8 listed on Table 2 for the presence of the deletion allele to generate B. thetaiotaomicron bt4554.

TABLE 2

| | | | Restriction |
|---|---|---|---|
| # | Primer | Sequence (5′→ 3′) | Site |
| 1 | Bt4554_D_F | ATATGGATCCGTGATGGATGGCATTCAGGC | BamHI |
| 2 | Bt4554_D_R_ | TTATTGTAAAACTAAAACCGTTCAATAACGA | |
| | SOE | TAATATATCCATCATAACAAAATGGCTTG | |
| 3 | Bt4554_U_F_ | CAAGCCATTTTGTTATGATGGATATATTATC | |
| | SOE | GTTATTGAACGGTTTTAGTTTTACAATAA | |
| 4 | Bt4554_U_R | ATATTCTAGATGATTCAACTCTTCCTGATGCG | XbaI |
| 5 | pEx 3240_F | GGAGAGGACGGACAGAAGATATAAACTC | |
| 6 | pEx 3611_R | CTCTCATGTTTCACGTACTAAGCTCTC | |
| 7 | Bt4554_vU_F | CTTTCTACTAAGATTGATTCTGAATCCGTACG | |
| 8 | Bt4554_vD_R | CAATGAAAAAACTCCCATCATTGTGAGC | |
| 9 | Bt4554_Cp_F | AACATTTAAAAAATAACATTCCATGAAAAAG | |
| | | TACTTTCCATC | |
| 10 | Bt4554_Cp_R | ACTGGAAGATAGGCAATTAGCTATATTCTGT | |
| | | CGAGTACAG | |
| 11 | pNBU_F | GCTGACATGGGAATTCC | |
| 12 | pNBU_R | CCATCACTGGAAGATAGG | |

The 300 bp upstream region of bt1311 (sigma 70; rpoD) in complementation vector pNBU2_erm_us1311 is replaced by each of 6 promoters (Table 3) conferring increasing transcriptional strength. Bt4554 was PCR-amplified (primers 9 and 10, Table 2), cloned into each of the constructed vectors (NEBuilder HiFi DNA Assembly Kit) and transformed into E. coli S17λ pir. Sequence-verified constructs (primers 11 and 12, Table 2) are introduced into B. thetaiotaomicron bt4554 by conjugation, generating B. thetaiotaomicron bt4554 pNP2E3_bt4554, B. thetaiotaomicron bt4554 pNP1E4_bt4554, B. thetaiotaomicron bt4554 pNP5E4_bt4554, B. thetaiotaomicron bt4554 pNP2E5_bt4554, B. thetaiotaomicron bt4554 pNP4E5_bt4554, and B. thetaiotaomicron bt4554 pNP1E6_bt4554 (in increasing order of promoter strength; plasmid names according to promoter designations discussed in W. R. Whitaker, E. S. Shepherd, J. L. Sonnenburg, Tunable Expression Tools Enable Single-Cell Strain Distinction in the Gut Microbiome. Cell. 169,538-546. 538-546.e12 (2017), the entire content of which is incorporated by reference herein).

TABLE 3

| Promoter | Sequence |
|---|---|
| P_BfP2E3 | gataaaacgaaaggctcagtcgaaagactgggcctttcg |
| | ttttacaattgggctaccttttttttgttttgtttgcaa |
| | tggttaatctattgttaaaatttaaagtttcacttgaac |
| | tttcaaataatgttcttatatgtgcagtgtcgaaagaaa |
| | caaagtag |
| P_BfP1E4 | gataaaacgaaaggctcagtcgaaagactgggcctttcg |
| | ttttacaattgggctaccttttttttgttttgtttgcaa |
| | tggttaatctattgttaacatttaaagtacacttgaact |
| | ttcaaataatgttcttatattttcagtgtcgaaagaaac |
| | aaagtag |
| P_BfP5E4 | gataaaacgaaaggctcagtcgaaagactgggcctttcg |
| | ttttacaattgggctaccttttttttgttttgtttgcaa |
| | tggttaatctattgttaaaatttaaagtacacttgaact |
| | ttcaaataatgttcttctatttgcagtgtcgaaagaaac |
| | aaagtag |
| P_BfP2E5 | gataaaacgaaaggctcagtcgaaagactgggcctttcg |
| | ttttacaattgggctaccttttttttgttttgtttgcaa |
| | tggttaatctattgttaaaatttaaagtttcacttgaac |
| | tttcaaataatgttcttatatttccagtgtcgaaagaaa |
| | caaagtag |
| P_BfP4E5 | gataaaacgaaaggctcagtcgaaagactgggcctttcg |
| | ttttacaattgggctaccttttttttgttttgtttgcaa |
| | tggttaatctattgttgaaatttaaagtttcacttgaac |
| | tttcaaataatgttcttatatttgcagtgtcgaaagaaa |
| | caaagtag |
| P_BfP1E6 | gataaaacgaaaggctcagtcgaaagactgggcctttcg |
| | ttttacaattgggctaccttttttttgttttgtttgcaa |
| | tggttaatctattgttaaaatttaaagtacacttgaact |
| | ttcaaataatgttcttatatttgcagTgtcgaaagaaac |
| | aaagtag |

2.7.4 Construction of Condensed Transposon Mutant Library

B. thetaiotaomicron mariner transposon insertion strains are selected from a library of 7155 B. thetaiotaomicron mutants, which are clonally arrayed and mapped by Insertion Sequencing (INSeq) as discussed in Goodman 2009. To maximize genome coverage with the smallest number of strains, mutants carrying multiple insertions and mutants with transposon insertions predicted to exhibit polar effects on downstream genes in the same operon are prioritized. Operons are predicted using a previously reported algorithm based on intergenic distances, conserved operon architecture and common functional annotation at a 90% confidence cutoff as discussed in B. P. Westover, J. D. Buhler, J. L. Sonnenburg, J. I. Gordon, Operon prediction without a training set. Bioinformatics. 21, 880-888 (2005), the entire content of which is incorporated by reference herein. After these filters are applied, strains carrying the most upstream insertion in each gene are selected and insertions in the last 10% of an ORF are not considered. This selection procedure results in a condensed library enriched for clones that i) carry insertions close to ORF start site as illustrated in FIG. 15A, ii) carry multiple transposon insertions as illustrated in FIG. 15B, and iii) disrupt multiple genes through polar effects as illustrated in FIG. 15C. FIGS. 15A-C relate to construction and characterization of a condensed B. thetaiotaomicron transposon insertion library. FIG. 15A illustrates distribution of transposon insertion relative position within each gene in the original (grey) and condensed (black) library. FIG. 15B illustrates number of insertions per strain in the original (grey) and condensed (black) library. Bars represent counts, normalized to the number of single insertions. Enrichment for multiple insertions resulted in about 50% of strains in the condensed library bearing more than one transposon insertion. FIG. 15C illustrates predicted number of genes perturbed by transposon insertions per strain in the original (grey) and condensed (black) library after selection of mutants that maximize predicted polar effects on downstream genes in the same operon. Bars represent counts, normalized to the number of insertions with transposons not predicted to cause polar effects.

Based on these criteria, 1290 insertions are selected that are predicted to collectively disrupt expression of 2350 unique genes. Selected strains are picked from frozen stocks of the source library as discussed in Goodman 2009 into 96-deep-well plates containing 0.5 mL of TYG medium. Each assayed plate contained several empty control wells (n=83) to monitor cross-contamination. After anaerobic incubation at 37° C. for 48 h, cultures are diluted (1:100) into TYG medium supplemented with erythromycin and gentamicin. After additional incubation for 36 hours, cultures are mixed with 40% glycerol (1:1) using a liquid handling robot (Eppendorf epMotion 5075) and stored at −80° C. until further use.

2.7.5 Enzyme Assays—Liver Assays of Conversion of BRV and SRV to BVU

Human and murine S9 liver fractions are purchased from Thermo Fisher Scientific (HMS9L and MSMCPL, respectively). Enzyme assays are performed for the deglycosylation of arabinosyluracil derivatives. In brief, assays are performed at 37° C. Reaction volumes are 150 μL with liver S9 fractions at 5 μg/μL and BRV or SRV at 100 μM in 10 mM phosphate buffer (pH 7.4). Reactions are initiated by addition of drugs to pre-warmed reaction mixture. 10 μL samples are collected and quenched in 10 μL acetonitrile on ice at 0, 5, 10, 15, 20, 30, 45, 60, 90, 120, 180, 270, and 360 min after initiation. Substrates and reaction product are extracted and quantified by LC-MS as described below.

2.7.6 Bacterial BRV Conversion Assays

All handling of human materials is conducted with the permission of the Yale Human Investigation Committee. Samples are collected and stored as described in A. L. Goodman et al., Extensive personal human gut microbiota culture collections characterized and manipulated in gnotobiotic mice, Proceedings of the National Academy of Sciences,108, 6252-6257 (2011), the content of which is incorporated by reference herein. In brief, a single fecal sample is collected from each healthy human donor, stored on ice for less than 12 hours prior to transport into an anaerobic chamber (Coy Laboratory Products) and homogenization in pre-reduced GMM containing 20% glycerol. Aliquots of 0.5 mL volume are anaerobically prepared in 1.8 mL glass E-Z vials (Wheaton Industries) and stored at −80° C. Murine fecal samples are collected from individually caged animals (2 males and 2 females) and are stored at −80° C. without further processing.

Frozen stocks are re-suspended in 4 mL pre-reduced GMM and incubated anaerobically at 37° C. for 14 h. Cultures are diluted (1:10) in 20% pre-reduced GMM containing BRV or SRV at 100 μM and further incubated at 25° C. anaerobically. 10 μL samples are collected and quenched in 10 μL acetonitrile on ice at 0, 15, 30, 45, 60, 90, 120, 180, 270, 360, 540, and 720 min after drug addition. Substrates and reaction product are extracted and quantified by LC-MS as described below.

Frozen stocks of bacteria (Table 4) are plated on BHI blood agar and incubated at 37° C. under anaerobic conditions. Single colonies are inoculated into 4 mL pre-reduced GMM and incubated anaerobically at 37° C. for 24 h (Akkermansia muciniphila for 48 hours). BRV conversion assays are performed with the resulting dense cultures, as described above for bacterial communities.

TABLE 4

| Name | Genotype or description | Reference |
|---|---|---|
| Bacteroides thetaiotaomicron | | |
| (Background: VPI 5482) | | |
| B. thetaiotaomicron | parent strain VPI 5482 | ATCC29148 |
| BT4554::Tn | bt4554::Tn | Goodman et al, |
| | | Cell Host |
| | | Microbe 2009 |
| wild type | Δtdk | Koropatkin et al, |
| | | Structure 2008 |
| bt4554 | Δtdk, Δbt4554 | This study |
| bt4554 pNBP2E3_BT4554 | Δtdk, Δbt4554, att1::P2E3_bt4554, | This study |
| | Erm^{R} | |
| bt4554 pNBP1E4_BT4554 | Δtdk, Δbt4554, att1::P1E4_bt4554, | This study |
| | Erm^{R} | |
| bt4554 pNBP5E4_BT4554 | Δtdk, Δbt4554, att1::P5E4_bt4554, | This study |
| | Erm^{R} | |
| bt4554 pNBP2E5_BT4554 | Δtdk, Δbt4554, att1::P2E5_bt4554, | This study |
| | Erm^{R} | |
| bt4554 pNBP4E5_BT4554 | Δtdk, Δbt4554, att1::P4E5_bt4554, | This study |
| | Erm^{R} | |
| bt4554 pNBP1E6_BT4554 | Δtdk, Δbt4554, att1::P1E6_bt4554, | This study |
| | Erm^{R} | |
| wild type* | Δtdk, att1, Erm^{R} | Degnan et al, Cell |
| | | Host Microbe 2014 |
| Other organisms | | |
| S17 | Escherichia coli S17, recA pro hsdR | ATCC47055 |
| | RP4-2-Tc::Mu-Km::Tn7 | |
| S17 pEx:bt4554KO | S17 pExchange_tdk:bt4554KO, Amp^{R} | This study |
| S17 pNBP2E3_BT4554 | S17 pNBU2_erm_P2E3_bt4554, Amp^{R} | This study |
| S17 pNBP1E4_BT4554 | S17 pNBU2_erm_P1E4_bt4554, Amp^{R} | This study |
| S17 pNBP5E4_BT4554 | S17 pNBU2_erm_P5E4_bt4554, Amp^{R} | This study |
| S17 pNBP2E5_BT4554 | S17 pNBU2_erm_P2E5_bt4554, Amp^{R} | This study |
| S17 pNBP4E5_BT4554 | S17 pNBU2_erm_P4E5_bt4554, Amp^{R} | This study |
| S17 pNBP1E6_BT4554 | S17 pNBU2_erm_P1E6_bt4554, Amp^{R} | This study |
| Bacteroides ovatus | strain: NCTC11153 | ATCC8483 |
| Prevotella copri | strain: 18205 | DSM18205 |
| Collinsella aerofaciens | strain: VPI 1003 | ATCC25986 |
| Clostridium symbiosum | strain: 2,M.Sebald LSU | ATCC14940 |
| Escherichia coli | strain: BW25113 | Yale Genetic |
| | | Stock Center |
| Akkermansia muciniphila | ATCC BAA-835 | ATCC BAA-835 |
| Plasmids | | |
| pExchange-tdk | Construct carrying cloned tdk (bt_2275), | Koropatkin et al, |
| | AmpR, ErmR | Structure 2008 |
| pEx:bt4554KO | pExchange-tdk with bt4554_SOE | This study |
| | deletion allele inserted | |
| pNBU2_erm | Suicide vector that inserts at att1/att2 | Koropatkin et al, |
| | sites, AmpR, ErmR | Structure 2008 |
| pNBU2_erm_bt1311 | pNBU2_erm with upstream promoter | Degnan et al, Cell |
| | region of bt_1311 for complementation | Host Microbe 2014 |
| pNBP2E3_bt4554 | pNBU2_erm_P2E3 with bt4554 | This study |
| pNBP1E4_bt4554 | pNBU2_erm_P1E4 with bt4554 | This study |
| pNBP5E4_bt4554 | pNBU2_erm_P5E4 with bt4554 | This study |
| pNBP2E5_bt4554 | pNBU2_erm_P2E5 with bt4554 | This study |
| pNBP4E5_bt4554 | pNBU2_erm_P4E5 with bt4554 | This study |
| pNBP1E6_bt4554 | pNBU2_erm_P1E6 with bt4554 | This study |

450 μL of GMM are inoculated with 50 μL of thawed B. thetaiotaomicron transposon mutant glycerol stocks and incubated anaerobically at 37° C. for 72 hours. Bacterial cultures (or GMM as a negative control) are diluted tenfold into 20% GMM containing 2 μM BRV and incubated anaerobically at 37° C. for 24 hours. 20 μL samples are collected over time for further processing and LC-MS analysis as described below.

2.7.7 Animal Experiments

All experiments using mice are performed using protocols approved by the Yale University Institutional Animal Care and Use Committee. Germfree (GF) 8 to 12 week old C57BL/6J mice are maintained in flexible plastic gnotobiotic isolators with a 12-hour light/dark cycle and GF status monitored by PCR and culture-based methods. Conventional C57BL/6J mice (Jackson Laboratories) are purchased at the age of 6-7 weeks and kept in the lab for 2-3 weeks before experiments. All mice are provided a standard, autoclaved mouse chow (5013 LabDiet, Purina) ad libitum.

Individually caged GF C57BL/6J mice are colonized by oral gavage with 200 μL of an overnight GMM culture of either B. thetaiotaomicron wild type or bt4554 strains to generate GN^{WT} or GN^{MUT} mice, respectively. After 4 days, bacterial loads are determined by CFU plating on BHI blood agar prior to drug treatment.

Each drug treatment is performed using 20 treated and 5 to 6 untreated animals. Both genders are equally represented in each group and evenly distributed across the different time points for sample collection. Animals (n=5 per time point and group) are given 100 mg/kg body weight of BRV or SRV as suspensions in PBS (200 μL PBS for controls) by oral gavage. One blood sample is collected from each animal at an early time point (0.5, 1, 1.5, or 2 hours after drug administration) by submandibular bleeding. At 3, 5, 7, and 9 h, mice are sacrificed and tissue samples are collected into sample tubes and snap-frozen. Fecal samples are collected before euthanization and re-suspended in PBS (1 mL) through vigorous shaking. 20 μL are then plated on BHI blood plates and incubated aerobically and anaerobically at 37° C. to check GF, GN^{WT}, and GN^{MUT} animals for contamination. Monocolonized mice are also checked for contamination by PCR using primers 7 and 8.

2.7.8 Sample Preparations for Drug and Metabolite Analysis

Liquid sample preparation is performed. In brief, 5 μl of internal standard solution (a mix of caffeine and sulfamethoxazole at 4 μM in H₂O) are added to each sample (20 μL) in 96-well plates (V-bottomed storage plate, Thermo Scientific) using a liquid handling robot (epMotion 5075, Eppendorf). Samples are extracted with 100 μL cold (−20° C.) organic solvent (acetonitrile:methanol, 1:1). After incubation for at least 1 hour at −20° C., samples are centrifuged (3220 rcf, −9° C.) for 15 min. 10 μL of supernatant were diluted with 10 μL H₂O for analysis by LC-MS. Extracted samples from the transposon mutant screen are dried for storage. To this end, 100 μL of supernatants are transferred to a new plate after organic extraction and centrifugation, dried under vacuum at 22° C. and stored at −80° C. For LC-MS analysis, the extracts are then resuspended in 6 μL methanol and further diluted with 26 μL H₂O.

200 μL of 0.1 mm zirconia/silica beads (BioSpec Products) and 500 μL of organic solvent (acetonitrile:methanol, 1:1) supplemented with internal standard are added to 50-350 mg of pre-weighed solid material. Material is homogenized by mechanical disruption with a bead beater (BioSpec Products) set for 2 minutes on high setting at room temperature. After incubation for at least 1 h at −20° C., samples are centrifuged (3220 rcf, −9° C.) for 15 min. 10 μL of supernatant are diluted with 10 μL H₂O for analysis by LC-MS.

2.7.9 LC-MS Quantification of Drugs and Metabolites

Samples for LS-MS analysis are prepared as described above. Chromatographic separation is performed on a C18 Kinetex Evo column (Phenomenex, 100 mm×2.1 mm, 1.7 mm particle size) using mobile phase A: H₂O, 0.1% formic acid and B: methanol, 0.1% formic acid at 45° C. 5 μL of sample are injected at 100% A and 0.4 mL/min flow followed by a linear gradient to 95% B over 5.5 min and 0.4 mL/min flow leading to thymine, BVU, SRV, and BRV elution at 0.95, 2.0, 2.15, and 2.4 min, respectively. The internal standards caffeine and sulfamethoxazole elute at 1.9 and 2.1 min, respectively. The qTOF is operated in positive scanning mode (50-1000 m/z) and the following source parameters: VCap is 3500 V; nozzle voltage is 2000 V; gas temp is 225 C; drying gas 13 L/min; nebulizer is 20 psig; sheath gas temp is 225 C; and sheath gas flow is 12 L/min. Online mass calibration is performed using a second ionization source and a constant flow (5 μL/min) of reference solution (121.0509 and 922.0098 m/z). Compounds are identified based on the retention time of chemical standards and their accurate mass (tolerance 20 ppm).

The MassHunter Quantitative Analysis Software (Agilent, version 7.0) is used for peak integration. Quantification is based on dilution series of chemical standards spanning 0.1 to 125 μM and measured amounts are normalized by weights of extracted tissue samples. Statistical analysis and plotting is performed in Matlab 2017b (MathWorks). Statistical significance of the differences between metabolite concentrations at each time point is assessed with Welch's t-test (unequal variances t-test, ttest2 function in Matlab).

2.7.10 Pharmacokinetic Multi-Compartment Modeling

The multi-compartment pharmacokinetic model, implemented by the prediction module 130, of drug metabolism in the mouse contained 7 compartments (small intestine I-III, cecum, colon, distal colon, and serum as illustrated in FIG. 19A). The serum compartment incorporates processes occurring in the liver, kidneys and all other body parts apart from the gastrointestinal (GI) tract. Exposure to the drug is modelled as an input to the small intestine I of the initial amount of drug equal to D*F, where D is the provided dose, and F is the bioavailability coefficient, and input to GI tract of the initial amount of D*(1−F). Drug propagation through the body is driven by the flow of GI material in different GI tract sections, compartment volumes and tissue: serum diffusion coefficients. Model parameters and equations are listed in Table 5. For BVU levels in serum, the BVU levels contributed by the host (due to host BRV metabolism) are distinguished from the BVU levels contributed by the microbiota (due to microbial metabolism in the cecum and BVU absorption). All equations are defined for drug and metabolite amounts. The model is created using the MatLab 2017b SimBiology Toolbox (MathWorks).

TABLE 5

| Parameter | Value | Units | Description |
|---|---|---|---|
| D | 9 | mmol | Drug was administered to reach |
| | | | concentration of 100 mg/kg; mouse |
| | | | mass 30 g, drug MW~333 => |
| | | | 100*30/333 = 9 mmol |
| F | 0.3 | fraction | Bioavailability of the drug |
| | | | (available for serum abSRVption); |
| | | | Clinical Virology 3rd edition, |
| | | | 2009, Douglas D. Richman and |
| | | | Richard J. Whitley |
| Vsi | 0.3 | mL | Volume of small intestine |
| Vserum | 10 | mL | Volume of distribution (measured |
| | | | in serum) |
| Vcecum | 3 | mL | Volume of cecum of germ-free mouse |
| Vcolon | 3 | mL | Volume of colon of germ-free mouse |
| | | | (cecal volume propagated to colon) |
| Vfeces | 3 | mL | Volume of feces of germ-free mouse |
| | | | (cecal volume propagated to feces) |
| VcecumCVR | 0.3 | mL | Volume of cecum of conventional mouse |
| VcolonCVR | 0.3 | mL | Volume of colon of conventional mouse |
| | | | (cecal volume propagated to colon) |
| VfecesCVR | 0.3 | mL | Volume of feces of conventional mouse |
| | | | (cecal volume propagated to feces) |

Model parameters are fit to the data using the sbiofit function in SimBiology toolbox in MatLab 2017 with the following parameters: globalMethod=‘ga’; hybridMethod=‘fminsearch’; hybridopts=optimset(‘Display’, ‘none’); options=optimoptions(options, ‘HybridFcn’, {hybridMethod, hybridopts}). Fitting is performed using the global optimization algorithm.

For the BRV propagation model, host drug metabolism parameters are fit to the data from GN^{MUT} mice. BRV measurements in small intestine I, cecum, colon, distal colon and serum, and BVU measurements in serum are converted from concentrations to amounts using volume estimates for each tissue. Microbial drug metabolism parameters are fit either to the data from GN^{WT} or CV mice. BRV measurements in cecum, and BVU measurements in cecum, colon and distal colon are used to fit the parameters.

For the SRV propagation model, host drug metabolism parameters are fit to the data from GF mice. SRV measurements in small intestine I, cecum, colon, distal colon and serum, and BVU measurements in serum are converted from concentrations to amounts using volume estimates for each tissue. Microbial drug metabolism parameters are fit to the data from CV mice. SRV measurements in cecum, and BVU measurements in cecum, colon and distal colon are used to fit the parameters.

In the combined host-microbiome drug metabolism model, BVU levels in the serum contributed by the host and the microbiota are predicted separately. For modeling of BRV metabolism, BVU serum levels contributed by the host are predicted based on host metabolism coefficients fitted to the data of GN^{MUT} mice administered BRV (host-only model). BVU serum levels contributed by gut microbes are predicted based on the microbial drug metabolism coefficient and BVU cecum absorption coefficients fit to the cecum and colon BVU data from GN^{WT} or CV mice administered BRV. For modeling of SRV metabolism, BVU serum levels contributed by the host after SRV exposure are predicted based on host metabolism coefficients fit to serum SRV data from GF mice. Serum BVU levels contributed by gut microbes after SRV administration are predicted based on the microbial drug metabolism coefficient and cecum BVU absorption coefficients fitted to the cecum and colon BVU data from CV mice administered SRV. For both BRV and SRV models, microbial contribution to serum BVU exposure is calculated as the ratio between areas under the curve of the microbial serum BVU levels and total serum BVU levels (the sum of microbial and host contributions).

A normalized sensitivity analysis is performed on the BRV model for total serum BVU exposure, serum BVU exposure contributed by the host, and serum BVU exposure contributed by gut bacteria. Model parameters fit for GN^{WT} mice are used as the base values. The influence of each parameter on serum BVU exposure is assessed by calculating the relative change in BVU exposure after changing each parameter in the range of 1% to 200% of the base value.

To investigate the influence of bioavailability (F), host drug to metabolite conversion coefficient, and microbial drug to metabolite conversion coefficient on the total BVU serum exposure and relative microbial contribution to serum BVU, the sbiosimulate function is used to determine the BRV model's behavior across different parameter values ranging from 0.01 to 0.99 for F, and 0.001 to 1000 in logarithmic scale for the conversion coefficients. For each model run, the area under the curve of BVU serum concentrations is calculated. The bacterial contribution is calculated as the ratio between microbial BVU absorbed from cecum to serum, and total BVU in the serum as illustrated in FIGS. 21A-L.

### 3. Advantage

The technology described herein may improve our understanding of the environmental and genetic factors that influence drug response variability.

The technology described herein provides an experimental approach to disentangle host and microbial contributions to drug metabolism, even in cases when host and microbial activities are chemically indistinguishable. Quantitative understanding of these host and microbiome-encoded metabolic activities as described herein clarify how nutritional, environmental, genetic and galenic factors impact drug metabolism and enable tailored intervention strategies to improve drug responses.

Existing approaches for predicting drug metabolism (computational or experimental) address host activities but do not provide any information about the microbiota. Prior art identifies drug-metabolizing host enzymes, but does not provide information about drug-metabolizing microbiota taxa. Prior art identifies only host genes that metabolize drugs. Prior art is limited to how human enzymes impact the metabolism of these other molecules. Prior art focuses on human genome polymorphisms (e.g. point mutations in CYPs), but does not provide any information on microbiota contributions. On the other hand, the technology described herein reveals the microbiome contribution.

The technology described herein does not target a specific disease indication, but instead is relevant to the development of any drug. Further, implementation of the technology described herein does not face the enormous cost and time statistics that apply to drug development.

Some estimates suggest that $1.4 billion and over 10 years is required per successful drug to reach the market. The technology described herein has the potential to reduce these statistics by identifying optimal drug candidates earlier in the development process. The technology described herein may allow appropriate early stage clinical trials to determine whether microbiome-mediated drug modification will impact drug safety and efficacy before reaching large and expensive Stage 3 trials. Accordingly, the technology described herein may reduce the high costs and high failure rate of pharmaceutical drug development.

The high-throughput approach of the present technology allows measurements of more than 20,000 candidate interactions per month. This is important because of the observed hit rate (˜3%). More broadly, the technology described herein identifies microbe and microbiome-mediated drug metabolism, which previous in silico, in vitro, and animal model approaches do not address.

The technology described herein also enables improved dosing and drug selection for therapeutics that are already approved for use.

The technology described herein can be applied to other molecules that are not drug candidates, including food components or food-derived molecules, other xenobiotics and other molecules. These measurements as described in the present technology provide the basis for a modeling framework that is generally applicable to biotransformations of other drugs, non-drug xenobiotics, food components and endogenous metabolites. The technology presented herein could be adapted for drugs converted to chemically distinct metabolites by the host and microbiome, and to other xenobiotics, food components and endogenous metabolites.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

Certain implementations of the disclosed technology are described above with reference to block and flow diagrams of systems and methods and/or computer program products according to example implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, or may not necessarily need to be performed at all, according to some implementations of the disclosed technology.

These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

Implementations of the disclosed technology may provide for a computer program product, comprising a computer-usable medium having a computer-readable program code or program instructions embodied therein, said computer-readable program code adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Additional examples for the identifications of drug-metabolizing genes from the microbiome, applications to predict metabolic activity from (meta)genomic sequences, and further pharmacokinetic models including the intestinal microbiome are provided in Zimmermann et al., Mapping Human Microbiome Drug Metabolism by Gut Bacteria and Their Genes, Nature (Jun. 3, 2019), available at https://www.nature.com/articles/s41586-019-1291-3, as well as in Zimmermann, Separating Host and Microbiome Contributions to Drug Pharmacokinetics and Toxicity, Science (Feb. 8, 2019), Vol. 363, Issue 6427, eaat9931, available at https://science.sciencemag.org/content/363/6427/eaat9931.

All patents, applications, publications, test methods, literature, and other materials cited herein are hereby incorporated by reference in their entirety as if physically present in this specification.

What is claimed is:

1. A system comprising:
a non-transitory storage medium storing information of levels of parent drug and drug metabolites for a plurality of oral drugs, by each of a plurality of microbiotas and by each of a plurality of microbes in pure culture; and
a processor in communication with the non-transitory storage medium, the processor configured to execute a predictor module which implements a computational model to perform the following:
quantitatively disentangling host and microbiota contributions to drug metabolism;
predicting how a subject's microbiota will metabolize a drug candidate;
predicting how the metabolization impacts the drug candidate and metabolite exposure in circulation; and
predicting whether the drug candidate will be metabolized by the microbiota.

2. The system of claim 1, wherein the non-transitory storage medium stores information of levels of parent drug and drug metabolites for each of 271 oral drugs, by each of 60 human gut microbiotas from unrelated human donors and by each of 76 defined and characterized (e.g. genome-sequenced) microbes in pure culture.

3. The system of claim 1, wherein the non-transitory storage medium stores measurements of drug and metabolite levels, collected over time and across tissues.

4. The system of claim 1, wherein the non-transitory storage medium stores information of a plurality of drug-microbiota interactions which reveal how microbes metabolize drugs.

5. The system of claim 1, wherein the non-transitory storage medium stores information of hierarchical clustering or other distance measurements of a set of microbes based on their ability to metabolize drugs, where related microbes are clustered together at broad and specific levels.

6. The system of claim 5, wherein the hierarchical clustering, based only on drug metabolism capacity, clusters related species from phylum to strain level, and clusters structurally similar drugs.

7. The system of claim 1, wherein the predicted drug activity includes at least one of pharmacogenomics and adverse effects.

8. The system of claim 1, wherein the processor performs clustering analysis to place chemically related drugs together based on their tendency to be metabolized by the same set of microbes.

9. The system of claim 1, wherein the microbiotas include gut microbiota.

10. The system of claim 1, wherein the microbes include archaebacteria or fungi.

11. The system of claim 1, wherein the processor determines what drug metabolites will be produced.

12. The system of claim 1, wherein the processor forecasts variation in drug response.

13. A system comprising:
a non-transitory storage medium storing information of levels of parent drug and drug metabolites for a plurality of oral drugs, by each of a plurality of microbiotas and by each of a plurality of genome-sequenced microbes in pure culture;
a processor in communication with the non-transitory storage medium, the processor configured to execute a predictor module which implements a pharmacokinetic model to perform the following:
receiving a microbiota composition as input; and
generating an output that predicts kinetics of microbiota-meditated metabolism of a drug candidate.

14. The system of claim 13, wherein the processor directly measures the kinetic constants of drug metabolism of a plurality of drugs and drug candidates by a plurality of individual microbiotas.

15. The system of claim 13, wherein the processor performs a high-throughput process for experimentally measuring whether and how many drug candidates are metabolized by a microbiota.

16. The system of claim 13, wherein the processor predicts whether and how the drug candidate will be metabolized by the microbiota.

17. The system of claim 13, wherein the processor predicts how inter-individual microbiota variations will impact how the drug candidate is metabolized.

18. The system of claim 17, wherein the processor predicts one or more of the following parameters of the drug candidate: toxicity and/or efficacy and/or pharmacokinetics.

19. The system of claim 13, wherein the processor identifies drug-metabolizing microbiota taxa for altering microbiota to achieve a lowest toxicity and highest efficacy for the drug candidate.

20. The system of claim 13, wherein the processor identifies microbial genes that confer specific drug metabolizing capabilities.

21. The system of claim 13, wherein the processor identifies individual genes in the microbiota that determine systemic levels of a toxic drug metabolite, and determines the toxicity of the drug candidate.

22. The system of claim 13, wherein the microbiota composition is defined by 16S rDNA sequencing or metagenomics.

23. The system of claim 13, wherein the processor receives chemical fingerprint of the drug candidate as input.

24. The system of claim 13, wherein the processor generates an output that estimates kinetic coefficient of metabolism for the drug candidate by the microbiota.

25. The system of claim 13, wherein the processor identifies a correlation between the microbiota composition and drug metabolism kinetics.

26. A system comprising:
a non-transitory storage medium storing information of levels of parent drug and drug metabolites for a plurality of oral drugs, by each of a plurality of microbiotas and by each of a plurality of genome-sequenced microbes in pure culture; and
a processor in communication with the non-transitory storage medium, the processor configured to execute a predictor module which implements a computational model to perform the following:
receiving a chemical structure of a drug candidate as input; and
predicting as output whether the drug candidate will be metabolized by each of the plurality of microbiotas and the microbes in the non-transitory storage medium.

27. The system of claim 26, wherein the processor generates an output that predicts whether and how the drug candidate will be metabolized by a microbiota.

28. A system comprising:
a non-transitory storage medium storing information of levels of parent drug and drug metabolites for a plurality of oral drugs, by each of a plurality of microbiotas and by each of a plurality of defined and characterized microbes in pure culture; and
a processor in communication with the non-transitory storage medium, the processor configured to execute a predictor module which implements a computational/pharmacokinetic model to perform the following:
predicting microbiota contribution to drug and metabolite exposure over time.

29. The system of claim 28, wherein the processor combines host-specific processes with microbiota-specific processes to predict how these processes influence the contribution of the microbiota to systemic drug and metabolite exposure.

30. The system of claim 29, wherein the host-specific processes include one or more of drug absorption and elimination, oral bioavailability, host metabolism and metabolite elimination.

31. The system of claim 29, wherein the microbiota-specific processes include one or more of intestinal transit, microbial metabolism, and metabolite absorption from the large intestine.

32. The system of claim 28, wherein the processor quantitatively predicts the contribution of gut microbiota to systemic drug and metabolite exposure, as a function of bioavailability, host and microbial drug metabolizing activity, drug and metabolite absorption, and intestinal transit kinetics.

33. The system of claim 28, wherein the microbes include genome-sequenced microbes.

34. A method comprising:
storing, by a non-transitory storage medium, information of levels of parent drug and drug metabolites for a plurality of oral drugs, by each of a plurality of microbiotas and by each of a plurality of genome-sequenced microbes in pure culture; and
executing, by a processor in communication with the non-transitory storage medium a predictor module which implements a computational model to perform the following:
quantitatively disentangling host and microbiota contributions to drug metabolism;
predicting how a subject's microbiota will metabolize a drug candidate;
predicting how the metabolization impacts the drug candidate and metabolite exposure in circulation; and
predicting whether the drug candidate will be metabolized by a microbiota.

35. A method comprising:
storing, by a non-transitory storage medium, information of levels of parent drug and drug metabolites for a plurality of oral drugs, by each of a plurality of microbiotas and by each of a plurality of defined microbes in pure culture;
executing, by a processor in communication with the non-transitory storage medium, a predictor module which implements a computational model to perform the following:
receiving a microbiota composition as input; and
generating an output that predicts kinetics of microbiota-meditated metabolism of a drug candidate.

36. The method of claim 34, wherein the processor generates an output that predicts kinetics of gut microbiota-meditated metabolism of a drug candidate.

37. A method comprising:
storing, by a non-transitory storage medium, information of levels of parent drug and drug metabolites for a plurality of oral drugs, by each of a plurality of microbiotas and by each of a plurality of defined microbes in pure culture; and
executing, by a processor in communication with the non-transitory storage medium, a predictor module which implements a computational model to perform the following:
receiving a chemical structure of a drug candidate as input; and
predicting as output whether the drug candidate will be metabolized by each of the plurality of microbiotas and the microbes in the non-transitory storage medium.

38. A method comprising:
storing, by a non-transitory storage medium, information of levels of parent drug and drug metabolites for a plurality of oral drugs, by each of a plurality of microbiotas and by each of a plurality of defined microbes in pure culture; and
executing, by a processor in communication with the non-transitory storage medium, a predictor module which implements a computational model to perform the following:
predicting microbiota contribution to drug and metabolite exposure over time.


SEQUENCE LISTING (18 sequences)

SEQ ID NO: 1 - DNA, 30 bp, Artificial Sequence
  Description of Artificial Sequence Synthetic primer
atatggatccgtgatggatggcattcaggc

SEQ ID NO: 2 - DNA, 60 bp, Artificial Sequence
  Description of Artificial Sequence Synthetic primer
ttattgtaaaactaaaaccgttcaataacgataatatatccatcataacaaaatggcttg

SEQ ID NO: 3 - DNA, 60 bp, Artificial Sequence
  Description of Artificial Sequence Synthetic primer
caagccattttgttatgatggatatattatcgttattgaacggttttagttttacaataa

SEQ ID NO: 4 - DNA, 32 bp, Artificial Sequence
  Description of Artificial Sequence Synthetic primer
atattctagatgattcaactcttcctgatgcg

SEQ ID NO: 5 - DNA, 28 bp, Artificial Sequence
  Description of Artificial Sequence Synthetic primer
ggagaggacggacagaagatataaactc

SEQ ID NO: 6 - DNA, 27 bp, Artificial Sequence
  Description of Artificial Sequence Synthetic primer
ctctcatgtttcacgtactaagctctc

SEQ ID NO: 7 - DNA, 32 bp, Artificial Sequence
  Description of Artificial Sequence Synthetic primer
ctttctactaagattgattctgaatccgtacg

SEQ ID NO: 8 - DNA, 28 bp, Artificial Sequence
  Description of Artificial Sequence Synthetic primer
caatgaaaaaactcccatcattgtgagc

SEQ ID NO: 9 - DNA, 42 bp, Artificial Sequence
  Description of Artificial Sequence Synthetic primer
aacatttaaaaaataacattccatgaaaaagtactttccatc

SEQ ID NO: 10 - DNA, 40 bp, Artificial Sequence
  Description of Artificial Sequence Synthetic primer
actggaagataggcaattagctatattctgtcgagtacag

SEQ ID NO: 11 - DNA, 17 bp, Artificial Sequence
  Description of Artificial Sequence Synthetic primer
gctgacatgggaattcc

SEQ ID NO: 12 - DNA, 18 bp, Artificial Sequence
  Description of Artificial Sequence Synthetic primer
ccatcactggaagatagg

SEQ ID NO: 13 - DNA, 164 bp, Artificial Sequence
  Description of Artificial Sequence Synthetic polynucleotide
gataaaacgaaaggctcagtcgaaagactgggcctttcgttttacaattgggctaccttttttttgttttgtttgcaatggttaatctattgttaaaatttaaagtttcacttgaactttcaaataatgttcttatatgtgcagtgtcgaaagaaacaaagtag

SEQ ID NO: 14 - DNA, 164 bp, Artificial Sequence
  Description of Artificial Sequence Synthetic polynucleotide
gataaaacgaaaggctcagtcgaaagactgggcctttcgttttacaattgggctaccttttttttgttttgtttgcaatggttaatctattgttaacatttaaagtttcacttgaactttcaaataatgttcttatattttcagtgtcgaaagaaacaaagtag

SEQ ID NO: 15 - DNA, 164 bp, Artificial Sequence
  Description of Artificial Sequence Synthetic polynucleotide
gataaaacgaaaggctcagtcgaaagactgggcctttcgttttacaattgggctaccttttttttgttttgtttgcaatggttaatctattgttaaaatttaaagtttcacttgaactttcaaataatgttcttctatttgcagtgtcgaaagaaacaaagtag

SEQ ID NO: 16 - DNA, 164 bp, Artificial Sequence
  Description of Artificial Sequence Synthetic polynucleotide
gataaaacgaaaggctcagtcgaaagactgggcctttcgttttacaattgggctaccttttttttgttttgtttgcaatggttaatctattgttaaaatttaaagtttcacttgaactttcaaataatgttcttatatttccagtgtcgaaagaaacaaagtag

SEQ ID NO: 17 - DNA, 164 bp, Artificial Sequence
  Description of Artificial Sequence Synthetic polynucleotide
gataaaacgaaaggctcagtcgaaagactgggcctttcgttttacaattgggctaccttttttttgttttgtttgcaatggttaatctattgttgaaatttaaagtttcacttgaactttcaaataatgttcttatatttgcagtgtcgaaagaaacaaagtag

SEQ ID NO: 18 - DNA, 164 bp, Artificial Sequence
  Description of Artificial Sequence Synthetic polynucleotide
gataaaacgaaaggctcagtcgaaagactgggcctttcgttttacaattgggctaccttttttttgttttgtttgcaatggttaatctattgttaaaatttaaagtttcacttgaactttcaaataatgttcttatatttgcagtgtcgaaagaaacaaagtag